(19) United States
(12) Patent Application Publication
(10) Pub. No.: US 2018/0311174 A1
(43) Pub. Date: Nov. 1, 2018

(54) NANOPARTICLES COMPRISING A METAL CORE SURROUNDED BY A MONOLAYER FOR LYMPH NODE TARGETING

(71) Applicants: Massachusetts Institute of Technology, Cambridge, MA (US); École Polytechnique Fédérale de Lausanne, Lausanne (CH)

(72) Inventors: Darrell J. Irvine, Arlington, MA (US); Yu-Sang Sabrina Yang, Cambridge, MA (US); Ahmet Bekdemir, Lausanne (CH); Francesco Stellacci, Morges (CH)

(21) Appl. No.: 15/770,442

(22) PCT Filed: Oct. 24, 2016
(86) PCT No.: PCT/US2016/058482

§ 371 Date: Apr. 23, 2018

Related U.S. Application Data
(60) Provisional application No. 62/245,845, filed on Oct. 23, 2015.

(51) Int. Cl. A61K 9/51; A61K 47/69

(52) U.S. Cl. CPC A61K 9/5115; A61K 47/6929; A61K 9/5123

(57) ABSTRACT

Amphiphilic gold nanoparticles (amph-NPs), composed of gold cores surrounded by an amphiphilic mixed organic ligand shell, are capable of embedding within and traversing lipid membranes. Active agent is bound thereto for use in vaccine and other adjuvant therapies, immunomodulation, and treatment of microbial infections, cancer, autoimmune disease, inflammation and inflammatory disorders.

### CROSS-REFERENCE TO RELATED APPLICATIONS

This application claims the benefit of and priority to U.S. Provisional Application Ser. No. 62/245,845, entitled LYMPH NODE TARGETED NANOPARTICLES, filed Oct. 23, 2015, the entire contents of which is incorporated by reference in its entirety.

### STATEMENT REGARDING FEDERALLY SPONSORED RESEARCH OR DEVELOPMENT

This invention was made with government support under grant No. W911NF-13-D-0001 (Project 6927308) from the U.S. Army. The government has certain rights in the invention.

### REFERENCE TO SEQUENCE LISTING

The Sequence Listing submitted as a text file named “MIT_18243_PCT_ST25.txt,” created on Oct. 24, 2016, and having a size of 1,034 bytes is hereby incorporated by reference pursuant to 37 C.F.R. § 1.52(e)(5).

### FIELD OF THE INVENTION

This invention is generally in the field of drug delivery platforms for delivery of agents to modify T cell activity, using nanoparticles targeted to the lymph nodes which are able to integrate into the cellular membranes and release agents therein.

### BACKGROUND OF THE INVENTION

Amplification of the immune system's ability to combat disease is a well-established clinical treatment strategy. The most effective means to stimulate immune activity is targeting lymph node resident lymphocytes directly, because lymph nodes are where most leukocytes reside. Efficient and targeted delivery of therapeutics to lymph nodes is desired for effective immunotherapy. This is achieved by lymph node-targeted delivery which minimizes peripheral absorption of pharmaceutical payloads. While intra-nodal delivery techniques are being developed (PNAS 2011, 108 (38), 15745-15750), under certain circumstances, for example, metastatic tumor, disruption of lymph node tissues might lead to cancer spread.

Immunotherapy is a highly efficacious treatment modality, however it is one fraught with challenges. While it promises to bolster the body's own defenses to combat infection, slight imperfections in immunotherapeutics can induce extreme local inflammation, systemic inflammation, or septic-shock. Any of these conditions can threaten major organs or be fatal. Systemic toxicity is often caused by pro or anti-inflammatory factors triggered at the dose required to produce the desired effect in the target tissue(s), rendering such treatments ineffective. Targeted delivery of precisely metered doses is therefore essential for developing to immunotherapeutics.

It is therefore an object of the present invention to provide compositions and methods of making and using the compositions for the delivery of hydrophobic, poorly water soluble molecules to the cytosol of cells.

It is another object of the present invention to provide compositions and methods of making and using the compositions for targeted delivery of therapeutic, prophylactic or diagnostic agents to lymph nodes, for preferential uptake by lymph node tissue, T cells, B cells, dendritic cells and/or macrophages.

### SUMMARY OF THE INVENTION

Amphiphilic gold nanoparticles (amph-NPs), composed of gold cores surrounded by an amphiphilic mixed organic ligand shell, are capable of embedding within and traversing lipid membranes. Active agent is bound thereto for use in vaccine and other adjuvant therapies, immunomodulation, and treatment of microbial infections, cancer, autoimmune disease, inflammation and inflammatory disorders. The amphiphilic nanoparticles can embed in the membrane of a variety of different cells. Because the nanoparticles target the lymph nodes, they embed at a higher frequency in lymph node-resident cells then in cells of other tissues. Embedding of the nanoparticle in the cell membrane drives efficient cytosolic delivery of whatever drug is to be delivered. As a result, the nanoparticles can be used for delivery of hydrophobic poorly water insoluble drugs to the cytosol of any drug and/or preferential delivery to the lymph nodes or lymph node resident cells such as dendritic cells, T cells and B cells. Additionally, or alternatively, such as peptides, can also be conjugated to the surface of the particle to facilitate their delivery to the lymph nodes.

A lymph-node targeting strategy has been developed that enables efficient drainage of nanoparticles (NPs) to both local and distal lymph nodes upon one single injection. Pharmaceuticals and/or vaccines that are targeted to lymphocytes may be loaded to these LN-targeted amphiphilic NPs (Amph-NPs) for better therapeutic outcome. Amph-NPs efficiently traffic in lymphatic systems and accumulate in lymph nodes for delivery of therapeutic, prophylactic and/or diagnostic agents to lymph node tissue, as well as dendritic cells (DCs), T-cells, and B-cells. Each nanoparticle core is surrounded by hydrocarbon chains terminated with sulfonate end groups and optionally pure hydrocarbon chains (Nature Materials 2008, 7 (7), 588-595).

The examples demonstrate that, following a single site tail base subcutaneous injection into mice, amph-NPs disseminate to both local and distal lymph nodes on both injection side and the opposite side. Nanoparticle concentration as high as 1414.2 μg/mg lymph tissue as well as 2.2% total injected NP per lymph node can be achieved using this method. Small molecule adjuvants loaded in amph-NPs delivered to mice stimulated immune response in lymph nodes with minimal systemic toxicity compared to adjuvants delivered alone in solution. Similarly, antigenic peptide-conjugated amph-NP's reduced tumor growth to a greater degree than free peptide in 5× excess.

### BRIEF DESCRIPTION OF THE DRAWINGS

FIG. 1 is a non-limiting illustration of amphiphilic gold (Au) nanoparticles with ligand shells formed from the ligands respectively shown below the NP: allMUS, MUSOT, PEG(4CH), PEG3k, and MPSA.

FIG. 2 is a non-limiting illustration of an amphiphilic gold NP being loaded with a hydrophobic therapeutic, prophylactic, or diagnostic agent (denoted by triangle shapes).

FIG. 3 is a schematic of amph-NP drug delivery and release from a cell membrane (adapted from Kim, et al., J Am Chem Soc., 131(4):1360-1 (2009)).

FIG. 4A is a graph of ICP-AES quantification (percentage of total injection) of Au NPs in lymph nodes 24 h post single site s.c. tail base injection as a function of ligand bound to the NPs: allMUS, MUSOT, PEG3k, and PEG(4CH). Au NPs solubilized in PBS and 50 μL of 6 mg/mL were injected subcutaneously on the left side of tail base. Lymph nodes were collected 24 h post injection and analyzed by ICP-AES. FIG. 4B is a graph of the percent total administered dose comparing levels of MUSOT Amph-NPs in the blood. FIGS. 4C-4G are bar graphs showing the organ distribution of the MUSOT Amph-NPs (left hand bar in each pair) and control PEGAu (right hand bar in each pair) in the lung (4C), liver (4D), spleen (4E), kidneys (4F), and bladder (4G), 4 or 24 hours after intravenous (i.v.) or subcutaneous (s.c.) injection. FIG. 4H shows that the MUSOT is 13× higher in the lymph nodes compared to PEGylated NPs.

FIGS. 5A-5B are dot plots showing distribution of MUSOT 100 μg, MUS 100 μg, MPSA 100 μg, PEG4CH 100 μg, PBS (5A); and MPSA, PGE (4CH), or PBS (5B) in natural killer cells (NK), T cells, B cells, macrophages, dendritic cells and neutrophils.

FIG. 6A is bar graph showing nanoparticle uptake in mouse lung cells (epithelial cell marker CD326− and CD326+) in vivo 24 h post intratracheal injection as measured by flow cytometry or CyTOF. FIGS. 6B and 6C are bar graphs showing the number of NPs per cell for FACS-sorted “Au low” cells subsequently analyzed by ICP-AES (6B) or CyTOF (6C). FIG. 6D is a histogram showing MUSOT amph-NP concentration in alveolar macrophages (MO) and F4/80 MΦ in the lung.

FIGS. 7A and 7B are dot plots quantifying drug loading capacity. DGKi loading in MUSPT, PEG(4CH) and MPSA NPs is shown in FIG. 7A. Drug loading of small molecules DGKi, R848, and TGF-beta inhibitor in amph-NPs of different core sizes is shown in FIG. 7B.

FIG. 8A is a diagram of macrophage-induced immunosuppression of T cells, and illustrates how DGK inhibitors can block the suppressive pathway prior to T cell dysfunction (adapted from thelancet.com/infection Vol 13 Mar. 2013). FIG. 8B is a graph of the % proliferating cells with or without PD-1L antibody and treated with (from left to right) vehicle, diacyl kinase inhibitor, diacyl kinase inhibitor-loaded amph-NPs, and amph-NP alone.

FIG. 9A is a plot showing the results of HPLC analysis of cytosolic TGF-β inhibitor concentration following delivery by amph-NPs relative to amph-NP control. FIG. 9B is a bar graph showing nanograms (ng) of TGF-β inhibitor per 2 million T cells treated with κ μg of TGF-β inhibitor in amph-NPs or 5 μg, 25 μg, or 50 μg of free drug.

FIGS. 10A-10D are dot plots of pg/ml of TNF (10A), IL-6 (10B), IL-10 (10C), and MCP-1 (10D) following treatment of cells with 10, 5, and 1 μg R848, R848-MUS, MUS, or PBS.

FIGS. 11A-11D are dot plots of showing the activation of dendritic cells (11A) and B cells and T cells: B220+CD3− cells (11B), CD3+CD8+ T cells (11C), CD3+CD4+ T cells (11D) via R848 (1, 5, 10 μg) delivered freely or with NPs.

FIG. 12A is an illustration of the structure of amph-NP with a peptide antigen (SEQ ID NO:2) attached thereto. FIG. 12B is a line graph showing the %-tetramer positive CD8+ T cells as a function of time after treatment with MUS/OT with SIINFEKL attached (SEQ ID NO:2), 5× SIINFEKL alone (SEQ ID NO:1), SIINFEKL construct alone (SEQ ID NO:2), or naïve. FIG. 12C is a bar graph showing the %-marker positive CD8+ T cells for markers IFN-γ+ and TNF-α+, IFN-γ+, and TNF-α+ after treatment with MUS/OT with SIINFEKL attached (SEQ ID NO:2), 5× SIINFEKL alone (SEQ ID NO:1), SIINFEKL construct alone (SEQ ID NO:2), or naïve. FIG. 12D is a line graph showing Tumor Area (mm²) as a function of time after treatment with MUS/OT with SIINFEKL attached (SEQ ID NO:2), 5×SIINFEKL alone (SEQ ID NO:1), SIINFEKL construct alone (SEQ ID NO:2), or naïve.

### DETAILED DESCRIPTION OF THE INVENTION

A nanoparticle lymph node delivery system is particularly efficacious in targeting the lymph system and cargos such as small molecules and peptides into the cytosol of lymph tissues and cells, as well as for lymphatic imaging and targeting specific cell types within the lymph system via antibody conjugation. The delivery system also has broad applications for cytosolic delivery of drugs, especially poorly water soluble hydrophobic drugs, to any cell type, including tumors. The system can also be used in vaccine-based applications to deliver antigens (e.g., peptide antigens), adjuvants (e.g., small molecule immunomodulators), or a combination thereof to cells in the lymph nodes.

No other approach to achieving lymph node targeting has been reported. PEGylation reduces non-specific protein adsorption to increase blood circulation half-life, however it lacks specificity in tissue targeting. As demonstrated by the examples using MUS and MUSOT amph-NPs, these are concentrated within the lymph system, but broadly distributed over the entire system. This can be used to dramatically reducing off-target cargo delivery of immunotherapeutics. The benefits are two-fold. First, the cargo itself is utilized more efficiently and administered in lower doses which can reduce the potential for adverse reactions while providing production cost savings to manufacturers. Second, adverse reaction risk is reduced by concentration of cargo delivery in the targeted tissue(s) or cell type(s) which allows for an increase in the maximum allowable clinical dose and thus expansion of therapeutic efficacy.

Small molecule cargo delivery is vastly superior to previous approaches due to: excellent NP/cargo-complex stability, hydrophobic cargo capability, and the predominance of membrane-transit release. These features open up entire classes of molecules for drug discovery. In vivo clearance and toxicity due to off-target absorption are mitigated. Efficacious treatments can be synthesized utilizing amph-NP vectors to deliver small molecule cargos.

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the term “nanoparticle”, generally refers to a particle having a diameter, such as an average diameter, from about 0.1 nm up about 100 nm. The nanoparticles can have any shape.

The term “amphiphilic”, as used herein, refers to a molecule combining hydrophilic and lipophilic (hydrophobic) properties.

As used herein, the term “ligand” refers to any molecule capable of forming a SAM wherein the molecules typically have three sections including an anchor, a tether, and an end group.

As used herein, the terms “self-assembled monolayers,” (SAMs) refer to monomolecular layers on surfaces. SAMs can be composed of ligands or a mixture of two or more ligands.

The term “Hydrophobic”, as used herein, refers to molecules which have a greater affinity for, and thus solubility in, organic solvents, as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

The term “Small Molecule”, as used herein, refers to a molecule, such as an organic compound, with a molecular weight of less than 2,000 Daltons, less than 1,500 Daltons, less than 1,000 Daltons, less than 750 Daltons, or less than 500 Daltons.

Numerical ranges disclosed herein disclose individually each possible number in such range, as well as any sub-ranges and combinations of sub-ranges encompassed therein. For example, a carbon range (i.e., C₁-C₁₀) is intended to disclose individually every possible carbon value and/or sub-range encompassed within. For example, a carbon length range of C₁-C₁₀ discloses C₁, C₂, C₃, C₄, C₅, C₆, C₇, C₈, C₉, and C₁₀, as well as discloses sub-ranges encompassed within, such as C₂-C₉, C₃-C₈, C₁-C₅, etc. Similarly, an integer value range of 1-10 discloses the individual values of 1, 2, 3, 4, 5, 6, 7, 8, and 10, as well as sub-ranges encompassed within. Further, a concentration range or weight percent range, such as from 1% to 2% by weight of the formulation discloses, the individual values and fractions thereof, such as 1%, 1.1%, 1.2%, 1.32%, 1.48% etc., as well as sub-ranges encompassed within.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₁₀ for straight chain, C₃-C₃₀ for branched chain), more preferably 20 or fewer carbon atoms, more preferably 12 or fewer carbon atoms, and most preferably 8 or fewer carbon atoms. In some embodiments, the chain has 1-6 carbons. Likewise, preferred cycloalkyls have from 3-10 carbon atoms in their ring structure, and more preferably have 5, 6 or 7 carbons in the ring structure. The ranges provided above are inclusive of all values between the minimum value and the maximum value.

The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls.

The alkyl groups may also contain one or more heteroatoms within the carbon backbone. Examples include oxygen, nitrogen, sulfur, and combinations thereof. In certain embodiments, the alkyl group contains between one and four heteroatoms.

“Alkenyl” and “Alkynyl”, as used herein, refer to unsaturated aliphatic groups containing one or more double or triple bonds analogous in length (e.g., C₂-C₃₀) and possible substitution to the alkyl groups described above.

“Aryl”, as used herein, refers to 5-, 6- and 7-membered aromatic rings. The ring may be a carbocyclic, heterocyclic, fused carbocyclic, fused heterocyclic, bicarbocyclic, or biheterocyclic ring system, optionally substituted as described above for alkyl. Broadly defined, “Ar”, as used herein, includes 5-, 6- and 7-membered single-ring aromatic groups that may include from zero to four heteroatoms. Examples include, but are not limited to, benzene, pyrrole, furan, thiophene, imidazole, oxazole, thiazole, triazole, pyrazole, pyridine, pyrazine, pyridazine and pyrimidine. Those aryl groups having heteroatoms in the ring structure may also be referred to as “heteroaryl”, “aryl heterocycles”, or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic.

“Alkylaryl”, as used herein, refers to an alkyl group substituted with an aryl group (e.g., an aromatic or hetero aromatic group).

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, containing carbon and one to four heteroatoms each selected from non-peroxide oxygen, sulfur, and N(Y) wherein Y is absent or is H, O, (C₁₋₄) alkyl, phenyl or benzyl, and optionally containing one or more double or triple bonds, and optionally substituted with one or more substituents. The term “heterocycle” also encompasses substituted and unsubstituted heteroaryl rings. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Heteroaryl”, as used herein, refers to a monocyclic aromatic ring containing five or six ring atoms containing carbon and 1, 2, 3, or 4 heteroatoms each selected from non-peroxide oxygen, sulfur, and N(Y) where Y is absent or is H, O, (C₁-C₈) alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

“Halogen”, as used herein, refers to fluorine, chlorine, bromine, or iodine.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms, such as nitrogen, may have hydrogen substituents and/or any permissible substituents of organic compounds described herein that satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

“Polymer”, as used herein, refers to a molecule containing more than 10 monomer units.

### II. Delivery System

A. Amphiphilic Nanoparticles (Amph-NPs)

Efficient NP drug delivery to lymph nodes may significantly enhance vaccine and immunotherapy outcomes. NP size, ligand chemistry, shape and charge all play important roles in their biodistribution and clearance (Nature Biotechnology 2015, 33 (9), 941-951). One major concern of introducing inorganic NPs to the clinics is their long-term accumulation in tissues. It is reported that NPs smaller than 5 nm are cleared out via renal route efficiently enough to be of interest as safe delivery vectors for clinical application (Biomaterials 2012, 33 (18), 4628-4638). Many strategies have been investigated to achieve targeted delivery. Experimentation with PEGylated nanoparticles has demonstrated reduced non-specific protein adsorption which increased blood circulation half-life (Nanomedicine 2011, 6 (4), 715-728). However, they do not accumulate in lymph nodes at a high rate.

The amphiphilic nanoparticles are valuable because they can intrinsically target lymph nodes and have excellent stability in post-synthesis storage and in vivo.

The amphiphilic nanoparticles have a core with a monolayer coating with at least a portion, more preferably the whole, of the surface of the nanoparticle useful for the loading of hydrophobic agents and as a potent and versatile delivery vector, especially to the lymph nodes.

Core Materials

The amphiphilic nanoparticles described herein have a core. The core can have a diameter of between 0.1 and 100 nm. The core may have a radius between about 1 and 10 nm, between about 1 and 25 nm, or between 1 and 50 nm. In certain embodiments, the core may be smooth or may be textured. Techniques well known to those skilled in the art may be used to impart texture to a surface, such as, but not limited to, plasma and chemical etching.

The nanoparticle core can be formed from materials including, but not limited to, gold, silver, platinum, palladium, copper, aluminum, a metal alloy thereof, or combinations thereof. In some embodiments, the nanoparticle core can be formed from materials including, but not limited to, silicon, silica, ceramics, alumina, a polymer, a semiconductor material, a composite of any of the aforementioned, or any suitable material onto which a SAM can attach to, or combinations thereof. Other materials, which are not ordinarily conducive to the formation of or attachment to a SAM, may be modified to render them more amenable to binding to an anchor group. For example, etching with a radio frequency (RF) oxygen plasma can establish hydroxyl groups at the surface of many materials, e.g., polymers that may be used to bind silanes or primary carboxylates to permit formation of a SAM. The preferred core material is gold.

Self Assembled Monolayers (SAMs)

The SAM coating present on the core of the nanoparticles includes a plurality of ligands which can form ordered domains having a characteristic size of less than or about equal to 10 nm. The portion of the nanoparticle surface, which may be textured and may have a radius of curvature of between about 0.1 and about 100 nm, between about 1 and about 5 nm, or between about 1 and about 10 nm. The ordered domains formed on the surface may be defined by morphologies which include, but are not limited to stripes, parallel stripes, strips, bands, ripples, a mosaic of roughly hexagonal domains on the portion, or combinations thereof. In other embodiments, the one or more domains formed are random. In yet embodiments, the domains formed are a mixture of ordered and random domains.

Ligands

Ligands may include any molecule capable of forming a SAM. In general, SAMs are formed of molecules having three sections including an anchor (A), a tether (T), and an end (E) group. A non-limiting embodiment of a ligand can have the formula A-T-E wherein the tether group interconnects the anchor and end groups. Ligands described herein can be prepared using methods known in the art.

The anchor group (A) can retain and/or bind the ligand on a substrate, such as a surface of the core of the nanoparticle. Each ligand may be connected to the portion of the nanoparticle surface by anchor group (A), which is, or contains, a moiety independently selected from, but not limited to, silane, carboxylate, thiol, phosphonate, nitrile, isonitrile, hydroxamate, acid chloride, anhydride, sulfonyl, phosphoryl, hydroxyl, and an amino acid. In some preferred embodiments, the anchor (A) group is a thiol. In some embodiments, the anchor group contains a single functionality therein that can attach to the surface, for example, an amine or dimethyl-methoxysilane moiety. Any art recognized anchor group (A) which can be used to anchor a ligand and form a SAM may be used in the formation of a monolayer coating on the particles. For example, organosilanes, carboxylic acids, sulfur-containing anchor groups, may be used as anchors. Metal cores formed from metals such as gold, silver, copper, cadmium, zinc, palladium, platinum, mercury, lead, iron, chromium, manganese, tungsten, and alloys of these may be patterned, for example, by forming thiol, sulfide, and disulfide bonds with ligands having sulfur-containing anchor groups (A). In addition, ligands may be attached to aluminum via a phosphonic acid (PO₃²⁻) anchor group. Nitriles and isonitriles, for example, may be used to attach molecules to platinum and palladium, and copper and aluminum may be coated with a SAM via a hydroxamic acid or hydroxamic acid-containing anchor group (A). Other functional groups suitable for use as anchors include, but are not limited to, acid chlorides, anhydrides, sulfonyl groups, phosphoryl and phosphonic groups, hydroxyl groups, and amino acid groups.

The tether group (T) is covalently attached to the anchor group (A). Any tether group which does not disrupt packing of the SAM and which preferably allows the SAM layer to be impermeable or substantially impermeable (“substantially impermeable,” as used herein refers to an ability to impede a reagent and/or solvent from passing through the SAM, such that at least 80%, 85%, 90%, 95%, 96%, 97%, 98%, or 99% of a reagent and/or solvent is precluded from passing through the SAM) to various reagents or organic or aqueous environments is suitable. The tether group may be polar, non-polar, halogenated (i.e., with fluorine), positively charged, negatively charged, or uncharged. Exemplary tethers groups (T) include, but are not limited to long chain (e.g., C₃-C₂₀ or more) hydrocarbon groups which may be optionally substituted. In some embodiments, the substituents of the hydrocarbon chain can be oxo, hydroxyl, carboxyl, amido or amino. For example, the tether (T) may be a saturated or unsaturated, linear or branched alkyl group or aromatic group. When the tether is an alkyl group it may also be interrupted one or more times by a heteroatom selected from oxygen, sulfur and nitrogen.

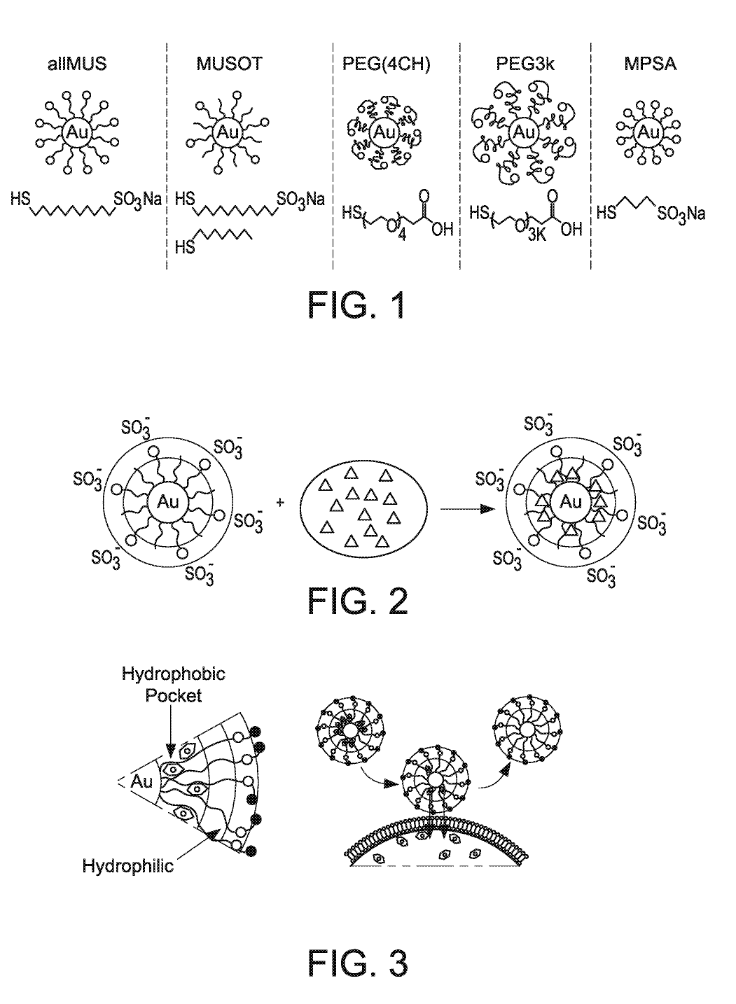

The end group (E) is attached to the tether group (T) and is connected to the anchor group (A) via tether group (T). The end group (E) is preferably exposed (i.e., point outwards from the monolayer attached to the core) when the SAM is formed (see FIG. 1). The end group (E) can be ionic, non-ionic, polar, non-polar, halogenated, alkyl, alkenyl, alkynyl, aryl or other functionalities which may be exploited as part of the end group. End groups with hydroxyl or amine moieties will tend to be hydrophilic, while halogenated and aliphatic groups will tend to be hydrophobic. Aromatic groups can contribute to specific chemical interactions and which may be photoactive. Alternatively, if no specific end group (E) is chosen, the end of the tether group (T) essentially forms the end group (E). For example, hydrocarbon tethers present a methyl end group, while a halogenated or hydroxylated hydrocarbon will present a halogenated or hydroxylated end group. The end group (E) may be hydrophobic or hydrophilic or selectively bind any one of various biological or other chemical species. A non-limiting, exemplary list of such end groups (E) include, but are not limited to: —OH, —CONH—, —CONHCO—, —NH₂, —NH—, —COOH, —COOR, —CSNH—, —NO₂—, —SO₂⁻, —RCOR—, —RCSR—, —RSR, —ROR—, —PO₄³⁻, —OSO₃⁻², —SO₃⁻, PO₃²⁻, NHₓR₄₋x⁺, —COO⁻, —SOO⁻, —RSOR—, —CONR₂, —SO₃H, —(OCH₂CH₂)ₙOH (where n=1-20), —CH₃, —PO₃H⁻, -2-imidazole, —N(CH₃)₂, —NR₂, —PO₃H₂, —CN, —(CF₂)—CF₃ (where n=1-20), olefins, hydrocarbons, etc. In some embodiments, the end group is —SO₃H or salts thereof; preferred salts include sodium and potassium salts. In some other embodiments, the end group is —CH₃. In the above list, R is hydrogen or an organic group such as a hydrocarbon or fluorinated hydrocarbon. As used herein, the term “hydrocarbon” includes aliphatic, aromatic, cyclic, polycyclic, unsubstituted, and substituted organics, e.g., alkyl, alkenyl, alkynyl, cycloalkyl, aryl, alkaryl, aralkyl, etc. The hydrocarbon group may, for example, comprise a methyl, propenyl, ethynyl, cyclohexyl, phenyl, tolyl, naphthyl, and benzyl group. The term “fluorinated hydrocarbon” is meant to refer to partially and fully fluorinated derivatives, in addition to perfluorinated derivatives of the above-described hydrocarbon groups.

Agent molecules may be attached to a SAM coating on the nanoparticle, such as through the ligands described above. For example, the end group (E) of the ligand may be reactive such that one or more agent molecules can be covalently or non-covalently linked to the ligand at the end group (E). Alternatively or in addition, the ligand may include a functional group that is capable of simulating a receptor that coordinates with an agent molecule.

Agent molecules which may be attached to the end group (E) via reaction with a functional group include, but are not limited to oligonucleotides, targeting moieties, polypeptides, antigens, dyes, MRI contrast agents, fluorophores, or small molecules, or combinations thereof. Oligonucleotides, polypeptides, dyes, MRI contrast agents, fluorophores, or small molecules, antigens, including cancer antigens, which may be used are known in the art. In some embodiments, the agent molecule may itself be substituted with another functional group. For example, the agent may be a peptide which is functionalized with a fluorophore. In a preferred embodiment, the ligand is an (N terminus) FITC-aminohexanoic acid (Ahx)-SIINFEKL-Ahx-cysteamide (C terminus) (SEQ ID NO:2).

The ligands described herein, containing an anchor group (A), tether group (T), and end group (E) forming a structure A-T-E (wherein the end group (E) may be optionally substituted with one or more agents) may be obtained, if not available from commercial sources, according to art known synthetic methods, to yield the desired ligand. Synthetic methodologies and strategies useful for the preparation of the ligands disclosed herein are known in the art. See, for example, March, “Advanced Organic Chemistry,” 5^{th} Edition, 2001, Wiley-Interscience Publication, New York).

Without limitation, an exemplary ligand can be prepared by reacting a tether group component, which contains an end group (E) (i.e., an amino group) and which also includes a reactive moiety (i.e., a carboxylic acid). An exemplary tether group containing an end group thereon is, for example, aminohexanoic acid. The tether group is then covalently attached to an anchor group or anchor containing group (i.e., a thiol containing group). For example, reacting aminohexanoic acid with a suitable amine-functionalized thiol-containing molecule, such as aminomethanethiol, standard amide bond-forming conditions (e.g., in the presence of a carbodiimide dehydrating agent, such as N,N′-dicyclohexylcarbodiimide (DCC), N,N′-Diisopropylcarbodiimide (DIC), or 1-Ethyl-3-(3-dimethylaminopropyl) carbodiimide (EDC) and a base, such as DMAP or triethylamine) may be used to form the following ligand:

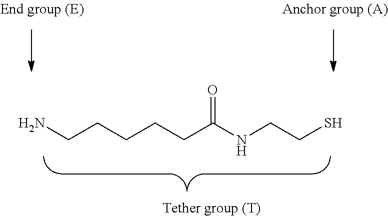 [SMILES: NCCCCCC(=O)NCCS]

In some embodiments, the terminal amino end group of the ligand may, for example, then be reacted with an agent containing a reactive group (such as a carboxylic acid group) to form an agent-functionalized ligand (i.e., Agent-E-T-A). In one non-limiting, such an agent-functionalized ligand may have the exemplary structure:

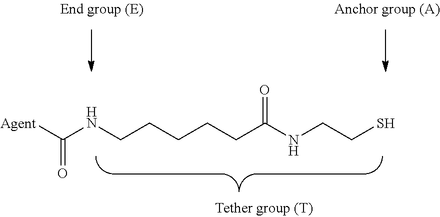

wherein the agent (see discussion above) is attached to the end group (E) of the ligand. In some embodiments, the preferred agent is a peptide, such as SIINFEKL (SEQ ID NO:1) and the functionalized ligand has the following non-limiting exemplary structure:

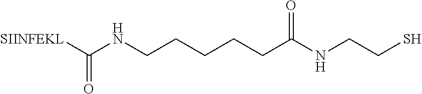

The agent may itself be optionally functionalized, prior to or following formation of the agent functionalized ligand, using art known techniques. In one non-limiting example, the agent is functionalized with functional group, such as a fluorophore (i.e., fluorescein isothiocyanate (FITC)), optionally through a linking molecule. An exemplary agent-functionalized ligand wherein the agent contains a fluorophore (FITC) bound thereto is:

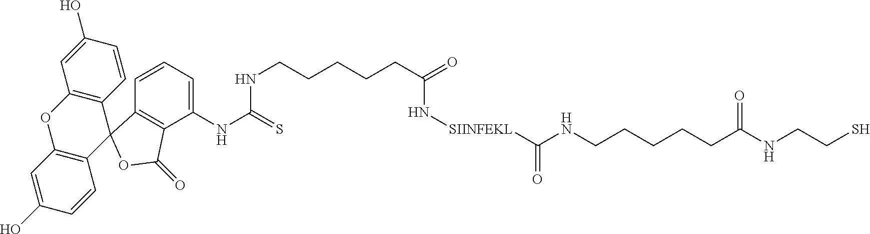

It will be appreciated that SIINFEKL (SEQ ID NO:1) in the foregoing formulas is being utilized for illustrative purposes and can be substituted with another peptide, peptide antigen, or another agent such those described in more detail elsewhere herein. Likewise, one or more of the depicted FITC, tether and anchor groups can also be substituted, replaced, or absent.

In yet another embodiment, agent molecules may be used as ligands themselves and attached directly to the surface of the core without the presence of a tether and/or end group. Such agents include, but are not limited to oligonucleotides, targeting moieties, polypeptides, antigens, dyes, MRI contrast agents, fluorophores, or small molecules, or combinations thereof which have been derivatized and/or functionalized to contain an anchor group, such as those described above. Non-limiting examples of such ligands would thus have the structure Agent-Anchor Group. In some embodiments, the agent of the Agent-Anchor Group ligand is a polypeptide. In some other non-limiting examples the ligands have the structure Agent-Cleavable Linker-Anchor Group; cleavable linkers are known in the art. Methods of derivatizing/functionalizing agent molecules with an anchor group (such as a thiol (—SH)), optionally linked via a cleavable group are known in the art.

The length of the tether (T) group, alone or in combination with end group (E) may be determined by certain factors, including the radius of curvature of the particle surface and the other ligand or ligands present in the mixture from which the SAM is formed. In one embodiment, the length of the ligand (A-T-E) is within about an order of magnitude of the radius of curvature of the nanoparticle surface. Where ligands are mixed in a ratio to form ordered domains (i.e., band-like or striped) on the nanoparticle surface, it may be undesirable to have one ligand be so much longer than the other ligand that it bends over and covers the second ligand.

The SAM monolayer coatings described herein are formed from such ligands described above. When more than one type of ligand is used, the difference in length of the ligands is typically less than the length of chain of 10 methylene groups.

Exemplary ligands, such as those described above, can independently be selected from, but are not limited to, mercaptopropionic acid, mercapto undecanoic acid, 4-amino thiophenol, hexanethiol, octanethiol, decanethiol, and duodecanethiol. In preferred embodiments, the ligands are selected from 11-mercaptoundecanesulfonic acid and salts thereof, 3-mercaptopropane-1-sulfonic acid and salts thereof, octanethiol, and mixtures thereof. In some embodiments, the ligands are selected from 11-mercaptoundecanesulfonic acid and salts thereof, 3-mercaptopropane-1-sulfonic acid and salts thereof, octanethiol, one or more agent-containing ligand (as described above), and combinations thereof.

The SAM monolayer coating may be formed from one or more ligands that, when deposited as self-assembled monolayers on a surface, exhibit contact angles with water that differ at least 1 degree, at least 3 degrees, at least 5 degrees, or at least 7 degrees. At least two members of a plurality of ligands may have differing hydrophilicities.

FIG. 1 is a non-limiting illustration of amphiphilic gold (Au) nanoparticles with ligand shells formed from the ligands respectively shown below the NP: allMUS, MUSOT, PEG(4CH), and PEG3k.

Targeting Moieties

The Amph-NP can also be modified to include a targeting moiety which may be associated with the Amph-NP non-covalently or attached to the core of the Amph-NP covalently by way of a ligand (i.e., attached to the end group of a ligand as discussed above). In some embodiments, the targeting domain includes all or part of an antibody that directs the particle to the desired target cell type or cell state.

In some embodiments, the targeting signal is used to selectively target tumor cells. Tumor cells express cell surface markers which may only be expressed in the tumor or present in non-tumor cells but preferentially presented in tumor cells. Exemplary tumor specific cell surface markers include, but are not limited to, alfa-fetoprotein (AFP), C-reactive protein (CRP), cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis(a)antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdr1 gene product), multidrug-resistance-related antigen, p170, multidrug-resistance-related antigen, prostate specific antigen (PSA), CD56, and NCAM. In some embodiments, the targeting signal consists of antibodies which are specific to the tumor cell surface markers.

In some embodiments, the targeting moieties target immune cells, such as macrophage, T cells, B cells, or dendritic cells. These are known to those skilled in the art.

Structural Features of Amphiphilic-NPs

The ligands will form domains on the surface of the nanoparticle defined by morphologies which include stripes, parallel stripes, strips, bands, ripples, a mosaic of roughly hexagonal domains on the portion, or combinations thereof. In yet other embodiments, the domains formed may be dis-ordered or random. In some embodiments, the domains formed are a mixture of ordered and disordered/random domains. The configuration of domains is dependent on the choice of ligands, the ligand ratio, and the nanoparticle curvature. Even where there is some mixing of the ligands within a domain, the distinct domains may still able to form.

Ordered domains may be formed from mixtures of ligands wherein the two or more ligands differ in length from each other. The difference in length need not be great and can be as small as one methylene group or other moiety in the chain (e.g., a secondary amine). It is not necessary that the ligands differ from one another in end group composition to form domains, although a difference in composition may be used to alter the properties of the SAM coated nanoparticles.

The relative ratios of the two or more ligands can determine the morphology of the domains formed. When two ligands are used to form a SAM, their molar ratio can be about 10:1, 9:1, 8:1, 7:1, 6:1, 5:1, 4:1, 3:1, 2:1, or 1:1. More evenly proportioned mixtures of ligands (for example, MUS:OT=2:1 or 1:1) can result in the formation of alternating stripes of each ligand.

In certain embodiments, the composition of the SAM monolayer (i.e., ligand-shell composition) is homogeneous and composed of 100% of a single type of ligand. In other embodiments, the ligand-shell composition is formed of two ligands in relative percentages, which may be about 99:1, 95:5; 90:10, 85:15, 80:20, 75:25, 70:30, 65:35, 60:40, 55:45, 50:50, 45:55, 40:60, 35:65, 30:70, 25:75, 20:80, 15:85, 10:90, 5:95, and 1:99. In some embodiments, the molar feed ratio of the two ligands in a range of about 99:1 to 1:99, or between any two values given above.

In the case of MUS:OT two ligand system, a preferred ligand ratio is about MUS:OT=2:1 or 1:1 for membrane-penetration properties. In these embodiments, the MUS ligand headgroup “sulfonate” provides hydrophilicity which is important for the high solubility of amph-NPs in water and under physiological conditions. It is believed that a ratio of MUS:OT below 1:1 (ex: 1:2 MUS:OT) may decrease NP solubility.

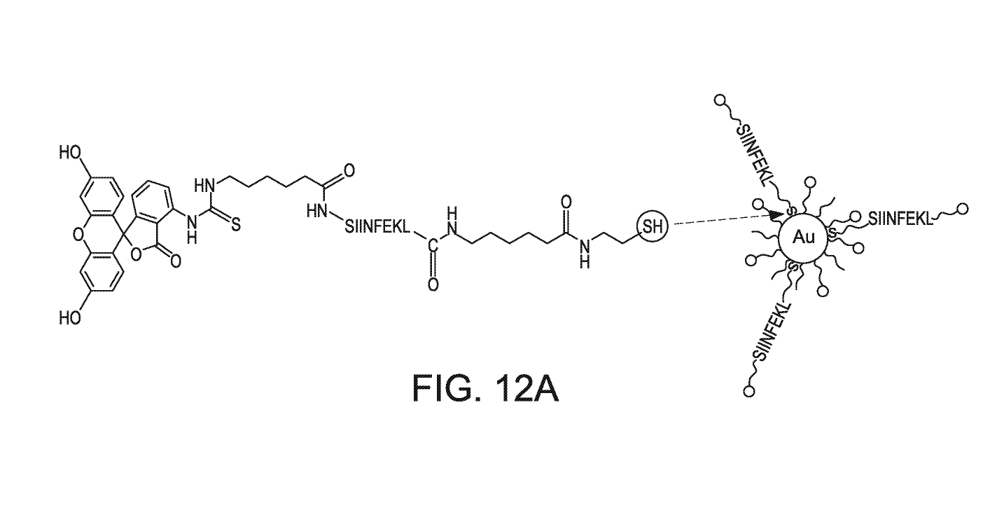

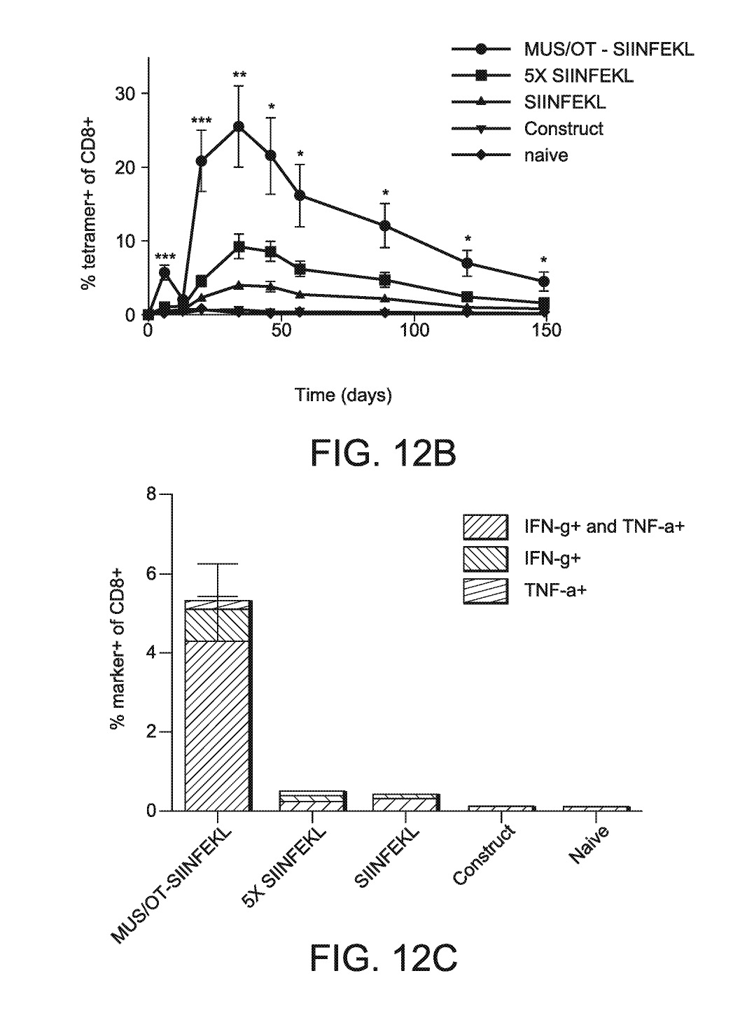

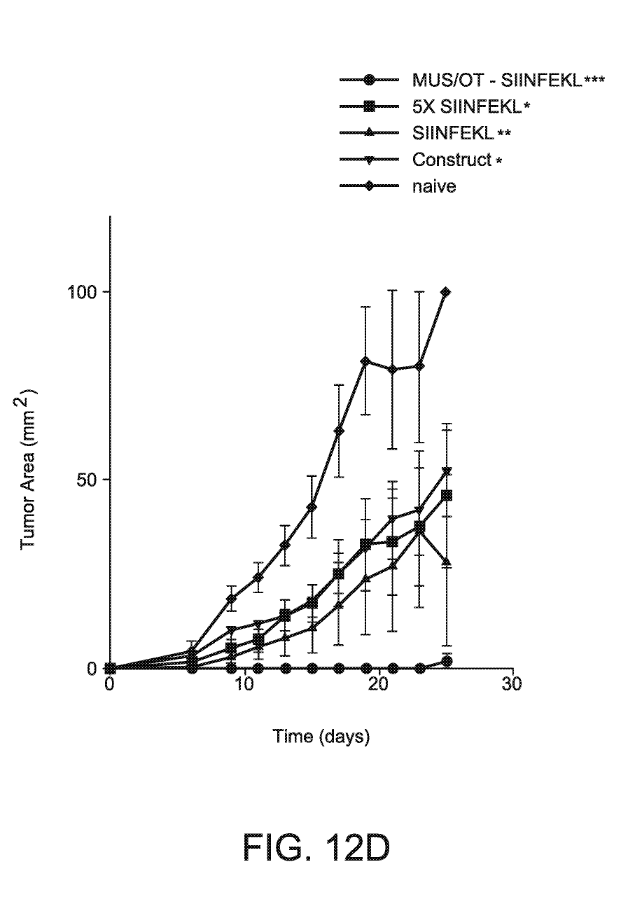

When the particles include an agent-based ligand such as a peptide antigen, it is generally desirable to strike a balance between getting enough functional groups attached for therapeutic effect but remaining/retaining enough amphiphilic surface property (MUS and optionally OT ligands) for cell-penetration and lymph node targeting. In an experimental example below (e.g., FIG. 12A) the particles included 3 ligands, wherein ˜7% of total original ligands (MUS AND OT) were replaced with Cys-Ahx-SIINFEKL ligands. This means ligand ratio of MUS:OT:SIINFEKL=9:4.5:1. Thus in some embodiments, the ratio of 3 ligands on the particle's surface is about 9 to about 4.5 to about 1 of MUS: OT: peptide.

In certain embodiments, a ligand mixture may be selected which includes both hydrophilic and hydrophobic ligands to afford alternating hydrophilic-hydrophobic domains. Such domains may be characterized by a width of between about 0.1 to 1 nm, about 1 nm to 2.5 nm, or 1 nm to 5 nm.

Synthesis of Nanoparticles

In a non-limiting exemplary synthesis of the amphiphilic nanoparticles described herein, the amphiphilic nanoparticles are formed from a mixture containing a metal salt and one or more of the ligands at suitable molar amounts and ratios. In one non-limiting example, a metal salt (such as HAuCl₄) is dissolved in a suitable solvent and the appropriate ligand mixture is added to the reaction mixture, followed by addition of a suitable reducing agent to afford the SAM coated amphiphilic nanoparticles, which are collected and washed to remove any residual unbound ligands. The particles may be purified by any suitable methods, such as dialysis, and optionally stored in solution or dried. Examples of making and characterizing amphiphilic nanoparticles are described in U.S. Pat. No. 7,597,950, Nature Materials 2008, 7 (7), 588-595, and Nano Lett. 2013, 13 (9), 4060-4067; which are incorporated in relevant part herein. Exemplary methods of characterizing the nanoparticles include, but are not limited to dynamic light scattering (DLS) to determine particle size and distribution, electron microscopy (i.e., scanning electron microscopy and transmission electron microscopy), and other relevant nanoparticle characterization techniques.

By varying the stoichiometry of the reagents used during a typical one-step synthesis, it is possible to control and change the height difference, the spacing and the shape of any phase-separated ordered domains which form on the exterior of the SAM coated nanoparticle. The global domain morphology can be controlled by varying the ligand ratio.

In some preferred embodiments, the amphiphilic nanoparticles are formed of a gold core and the SAM monolayer on the nanoparticle core is formed of a salt of mercaptoundecanesulfonic acid (i.e. sodium mercaptoundecanesulfonate) or, alternatively formed from a mixture of a salt of mercaptoundecanesulfonic acid and octanethiol using the methods described above. In some embodiments, the SAM is formed from a mixture of a salt of mercaptoundecanesulfonic acid (MUS), octanethiol (OT), and an agent-functionalized ligand (as described above and in example 5).

In some embodiments, the amphiphilic nanoparticles described herein may be labeled with a fluorescent agent. For example, a thiolated BODIPY dye as described in Nature Materials 2008, 7 (7), 588-595 may be used to label the nanoparticles.

Methods of Loading Hydrophobic Agents in Amph-NPs

The amphiphilic nanoparticles are loaded with small molecule hydrophobic agents. Hydrophobic regions present in the SAM on the NP (i.e., the ligand shell) allow for an energetically favorable temporary storage location for small hydrophobic molecules. Upon delivery to one or more target cells or tissues thereof, trans-membrane passage results in disturbance of the ligand shell and release of at least some of the hydrophobic agents.

In a non-limiting example of the loading method, hydrophobic agents in a suitable organic solvent are mixed with an aqueous suspension of the amphiphilic nanoparticles and dialyzed to remove the organic solvent which leads to partitioning of the drug into the hydrophobic regions and/or pockets of the amphiphilic nanoparticle ligand shell. This approach allows loading of hydrophobic agents that are nearly insoluble in aqueous solutions to high degrees of “solubility” via incorporation into the amphiphilic nanoparticles.

Another exemplary procedure to prepare hydrophobic agent-loaded nanoparticles includes: 1) Hydrophobic agents are dissolved in an alcohol such as pure ethanol; 2) mixed with the nanoparticles in a dialysis tubing against water or other suitable solvent (dialysis tubing containing a 10-500 Da molecular weight cut off allows ethanol to permeate while retaining most small molecules, although higher cut off weights can also be used), 3) Removal of ethanol from mixture such that hydrophobic agents are driven into hydrophobic ligand shells of amphiphilic nanoparticles to minimize unfavorable interaction with water, and 4) collecting the resulting dialyzed to solutions from the dialysis tubes and removing the solvent was removed under vacuum, preferably at 45° C. or less.

FIG. 2 is a non-limiting illustration of an amphiphilic gold NP being loaded with a hydrophobic therapeutic, prophylactic, or diagnostic agent (denoted by triangle shapes).

The degree of agent loading can be quantified via absorbance measurements using UV-vis spectrometer.

B. Therapeutic, Prophylactic and Diagnostic Agents to be Delivered

Many small molecules are under intensive investigation as new pharmaceuticals for cancer and infection treatments. They are potent, have well-defined structures and are often cost effective. However, many of them are not soluble in water and have intolerable off-target toxicity. If a hydrophobic molecule cannot traverse the milieu of aqueous environments and membranes enroute to its cytosolic target, then the drug cannot be effective. While in vitro testing of various new hydrophobic small molecules can demonstrate desirable physiological effects, the same molecules generally suffer from exceptionally rapid clearance in vivo. Doses required to achieve the observed in vitro effects often cause systemic toxicity. Being able to deliver concentrated small molecules via NPs to targeted sites would solve the issues associated with their soluble form.

Active agents include, for example, anti-cancer agents such as, but not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), and combinations thereof. In one embodiment, the preferred compounds are hydrophobic compounds with low rates of encapsulation using standard polymeric encapsulation. The NPs described herein show high drug loading than with standard polymeric encapsulation. In another embodiment, preferred compounds are those with low rates of uptake into the cell cytosol. The NPs described herein show higher rates of uptake of these compounds into the cell cytosol as compared to standard polymeric encapsulation (i.e., using an emulsion technique).

The active agent can be a small molecule immunomodulator. Such drugs are known in the art and are gaining attention, particularly in the field of cancer treatment. For example, drugs that can antagonize surface enzyme-linked receptors and receptors that interact with the tumor microenvironment, or that even inhibit metabolic enzymes, have been shown effective for inducing or modulating immune response against cancer (Iyer, et al., Anti-Cancer Agents in Medicinal Chemistry, 15(4): 433-452 (2015)). Molecules have also been identified that can directly inhibit the signaling initiated by the respective ligands binding to their receptors, recruit antibodies and other immunomodulatory molecules, and promote or inhibit the proliferation of different immune cells to target specific types of cancer cells. Small molecule immune response modifiers are known in the art and include, for example, imiquimod, antibody-recruiting molecules that target prostate cancer, integrin receptor antagonists, indoleamine-2,3-dioxygenase inhibitors, emodin, RORγt antagonists, ephrin receptor antagonists, membrane-bound carbonic anhydrase IX (CAIX) inhibitors, selected protein kinase inhibitors, and others reviewed in (Iyer, et al., Anti-Cancer Agents in Medicinal Chemistry, 15(4): 433-452 (2015), which is specifically incorporated by reference herein in its entirety. See also, Failli and Caggiano, “Patent Update: Small Molecule Immunomodulators,” Expert Opinion on Therapeutic Patents, 2(6):882-892 (2011). DOI: 10.1517/13543776.2.6.882). In particularly preferred embodiments, the active agent is a small molecule that acts as Toll-Like Receptor (TLR) agonist.

The active agent can also be an immunosuppressant. Immunosuppressants can be used to treat autoimmune disease, inflammation, graft verse host disease, and to prevent graft rejection during transplantation. Small molecule immuosuppressants include, but are not limited to, glucocorticosteroids, immunophilin-binding drugs, calcineurin inhibitors (e.g., CsA, TAC), target of rapamycin inhibitors (e.g., sirolimus, RAD), inhibitors of de novo nucleotide synthesis including purine synthesis (e.g., IMPDH inhibitors such as mycophenolic acid, mycophenolate mofetil, mizoribine), pyrimidine synthesis (e.g., DHODH inhibitors such as brequinar, beflunomide), azathioprine, antimetabolites, steroids, anti-proliferatives, and cytotoxic agents, (Medscape Multispecialty, “Molecular Mechanisms of Immunosuppressive Drugs and Their Importance in Optimal Clinical Outcomes” (accessed October 2015)). Particular immunosuppressants include, but are not limited to, methotrexate, cyclophosphamide, deoxyspergualin and related compounds, FTY720, cyclosporin A, FK506-like compounds, and rapamycin compounds.

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds include, for example, those described in WO 00101385. Preferably, the language “rapamycin compound” as used herein does not include FK506-like compounds.

Other suitable therapeutics include, but are not limited to, anti-inflammatory agents. The anti-inflammatory agent can be non-steroidal, steroidal, or a combination thereof. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

The active agent can be an antibiotic. Exemplary antibiotics include, but are not limited to, aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim). Other compounds include antiviral and anti-parasitic compounds, including anti-malarial compounds.

For imaging, radioactive materials such as Technetium99 (^{99m}Tc) or magnetic materials such as γ-Fe₂O₃ can be used. Examples of other materials include gases or gas emitting compounds, which are radiopaque, and fluorophores.

Fluorophores are fluorescent chemical compounds that can re-emit light upon light excitation. Fluorophores are well known in the art and include, but are not limited to, acridine derivatives (proflavin, acridine orange, and acridine yellow), anthracene derivatives (e.g., anthraquinones, including DRAQ5, DRAQ7 and CyTRAK Orange), arylmethine derivatives (e.g., auramine, crystal violet, and malachite green), coumarin derivatives, cyanine derivatives (e.g., cyanine, indocarbocyanine, oxacarbocyanine, thiacarbocyanine, and merocyanine), naphthalene derivatives (dansyl and prodan derivatives), oxadiazole derivatives (e.g., pyridyloxazole, nitrobenzoxadiazole and benzoxadiazole), oxazine derivatives (Nile red, Nile blue, cresyl violet, and oxazine 170), pyrene derivatives (e.g., cascade blue), squaraine derivatives and ring-substituted squaraines (e.g., Seta, SeTau, and Square dyes), and tetrapyrrole derivatives (e.g., porphin, phthalocyanine, and bilirubin), and xanthene derivatives (e.g., fluorescein, rhodamine, Oregon green, eosin, and Texas red).

In some embodiments, particles are loaded with LCL161, an IAP inhibitor that stimulates T cell function in cancer. This small molecule is hydrophobic and loads well in amph-NPs. In some embodiments, particles are loaded with SN-38, an anti-cancer cytotoxin that inhibits DNA topoisomerase I.

In some embodiments, the particles are used to deliver one or more oligonucleotides (e.g., single or double stranded DNA, RNA, peptide nucleic acids, locked nucleic acids, etc.), polypeptides, dyes, MRI contrast agents, fluorophores, small molecules or combinations thereof by attaching it/them to the particle or a component of the particle as discussed in more detail above and exemplified below. Some such embodiments have one or more small molecules entrapped in the SAM monolayer or ligand layer. In this way two or more different types of cargo/agent can be delivered to the same cell at the same time. In some other embodiments, the particle includes one or more ligands, but no small molecule is entrapped in the SAM monolayer or ligand layer. As discussed in more detail below, in some embodiments, two or more different types, species, or forms of particles are delivered together in the same or different admixtures.

Dosing

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual polynucleotides, and can generally be estimated based on EC50s found to be effective in vitro and in vivo animal models. In some embodiments, less of the molecule or molecules being delivered by the particle can be used when delivered by the particle relative to when delivered as free molecule(s).

In some embodiments, the effect of the composition on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects).

In some embodiments, the effect of the treatment is compared to a conventional treatment that is known the art, such as one of those discussed herein. Preferably, the disclosed compositions have less toxicity than free molecule at the same dosage, a greater potency or other pharmacological effect than free molecule at the same dosage, or a combination thereof. In some embodiments, the compositions can be administered at a lower dosage than free molecule, but achieve a greater therapeutic effect, lower toxicity, or a combination thereof.

Dosage levels on the order of about 0.01 mg/kg to 100 mg/kg or 0.05 mg/kg to 50 mg/kg or 0.1 mg/kg to 10 mg·kg of body weight per administration are useful in the treatment of a disease. One skilled in the art can also readily determine an appropriate dosage regimen based on the known pharmacokinetics of the agents delivered using standard delivery.

For example, 0.15 mg/kg of ciprofloxacin was effective in early eradication of local pseudomonas infection when delivered with amph-NPs, whereas the same dose ciprofloxacin delivered freely (without NP) had minimal effect in infection clearance.

Loading efficiency is a function of ligand properties, core size as well as a drug's size/hydrophobicity/charge, and the examples below show that the amount of drug per particle is tunable by adjusting the length, ratio, and/or species of SAM components as well as the size of the core. In some embodiments, the drug is a loaded at molar ratio of between about 1:1 and about 1,000:1. Loading of several drug molecules per NP at a molar ratio of 5:1, 10:1, 20:1, 30:1, 40:1, 50:1 all the way up to 350:1 has been empirically confirmed. Thus in some embodiments the drug per particle is loaded at a molar ratio of about 5:1, about 10:1, about 20:1, about 30:1, about 40:1, about 50:1 about 75:1, about 100:1, about 150:1, about 200:1, about 250:1, about 300:1, about 350:1, about 400:1, about 450:1, or about 500:1.

C. Formulations

Pharmaceutical compositions can be for administration by parenteral (intramuscular, intraperitoneal, intravenous (IV) or subcutaneous injection), or transmucosal (nasal, pulmonary, vaginal, rectal, or sublingual) routes of administration and can be formulated in dosage forms appropriate for each route of administration. The compositions are most typically administered systemically.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients.

Compounds and pharmaceutical compositions thereof can be administered in an aqueous solution, suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include as diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration, by incorporating sterilizing agents into the compositions, by irradiating the compositions, or by treatment with ethylene oxide gas.

In one embodiment, the compositions are formulated for pulmonary delivery, intranasal administration or application to a mucosal surface (oral, vaginal, or rectal). A number of compounds are approved for pulmonary administration, including small molecules such as those used to treat asthma as well as particles such as insulin particles. Nasal delivery is considered a promising technique for administration of therapeutics since the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

### III. Methods of Use

The amph-NP can be used to deliver a therapeutic, prophylactic and/or diagnostic agent to the cytosol of any cell. It can also be targeted to the lymph nodes for preferential uptake by lymph tissue, including lymph resident cells. Because the amph-NP home to the lymph nodes, in some embodiments they are used to deliver cargo to lymph node-resident cells at a higher frequency then in cells of other tissues. Lymph resident cells can include immune cells such lymphocytes (B cells, T cells, natural killers cells) and other lymphocytes. In some embodiments, the amph-NP is used to deliver a therapeutic, prophylactic and/or diagnostic agent to the cytosol of non-resident or diseased cell found within the lymphatic system, for example, cancers or virally infected cells. FIG. 3 is schematic of amph-NP drug delivery and release from a cell membrane (adapted from Kim, et al., J Am Chem Soc., 131(4):1360-1 (2009)).

Exemplary therapeutic uses are discussed in more detail below and exemplified in the working examples. Small molecule adjuvants delivered via LN-targeted NPs activated local immune cells with minimal systemic toxicity. By leveraging the intrinsic lymph node targeting and small molecule cargo capacity, the nanoparticles can be used to deliver small molecule adjuvants to lymph-node resident immune cells.

An exemplary workflow for making and using amph-NP can including preparation of membrane-embedding gold NPs, hydrophobic drug loading onto the gold NPs, drug delivery to cells in vitro or in vivo, and accumulation in the lymph nodes.

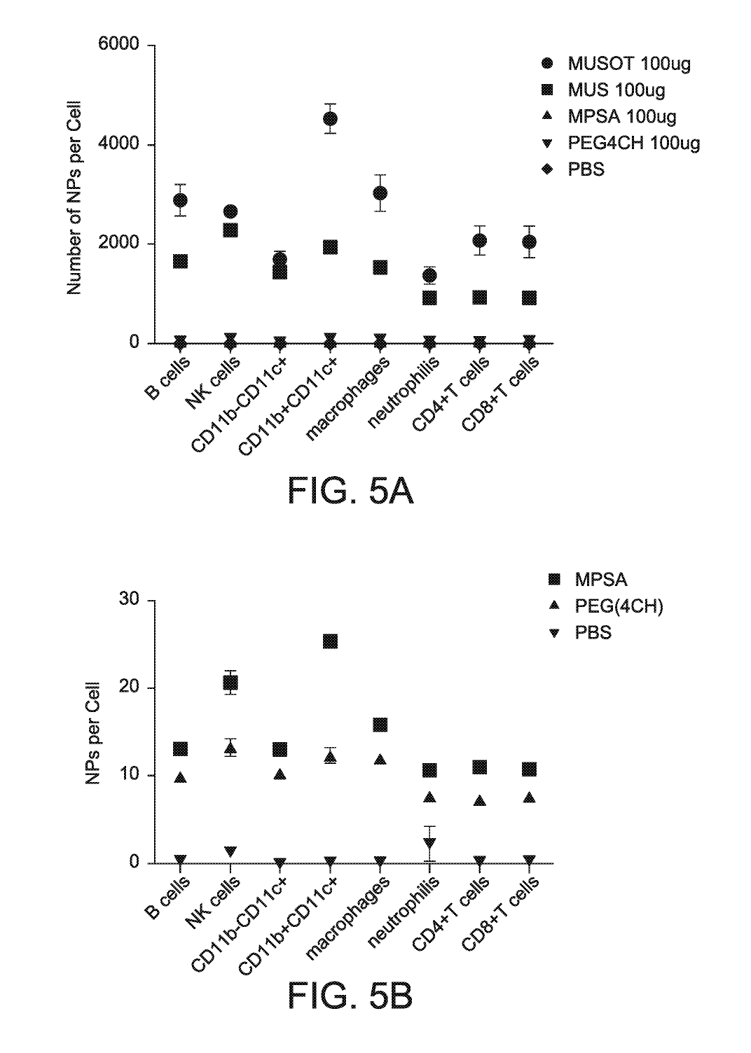

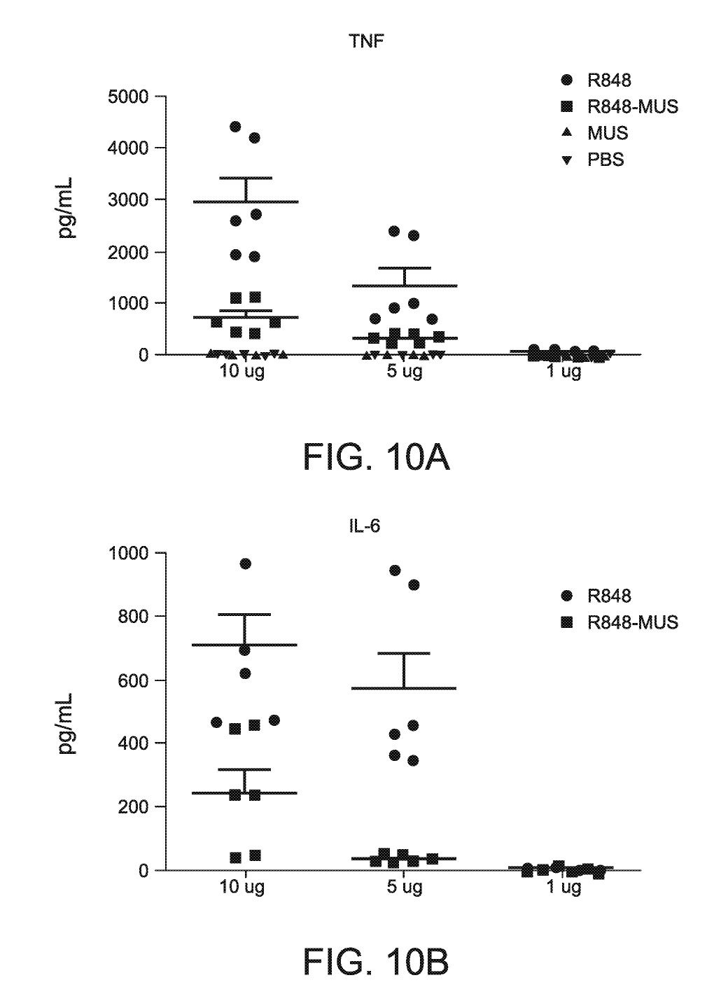

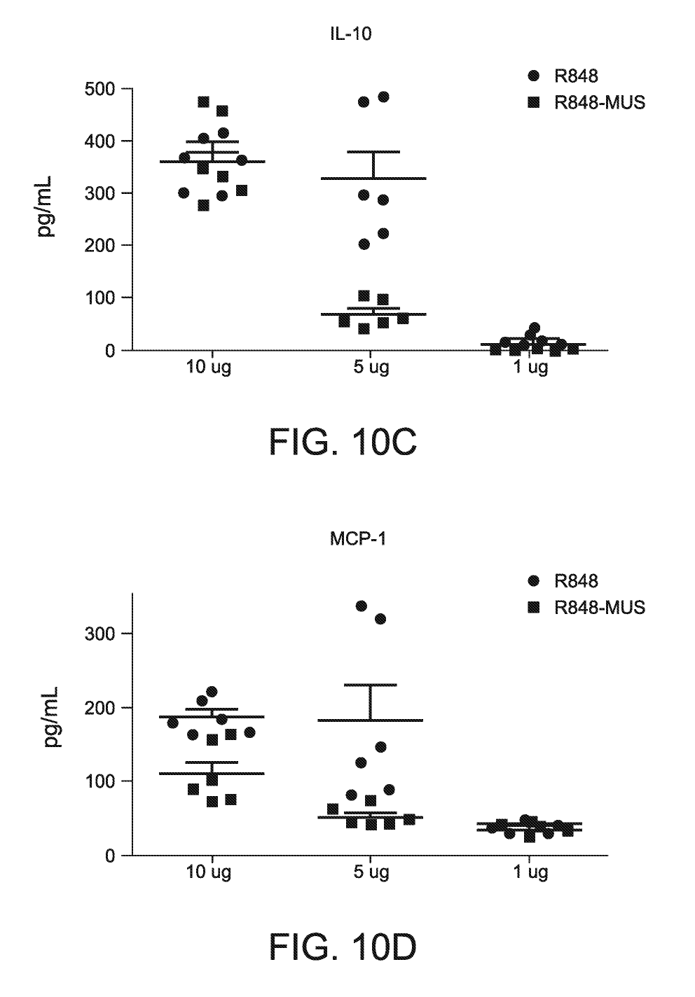

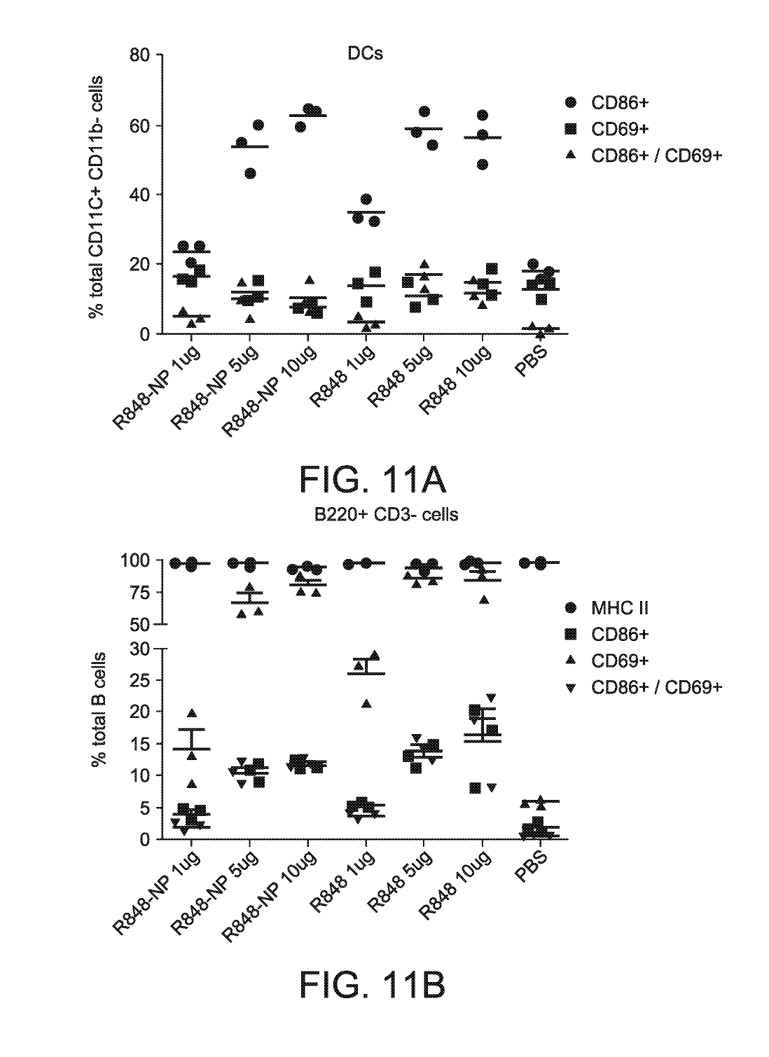

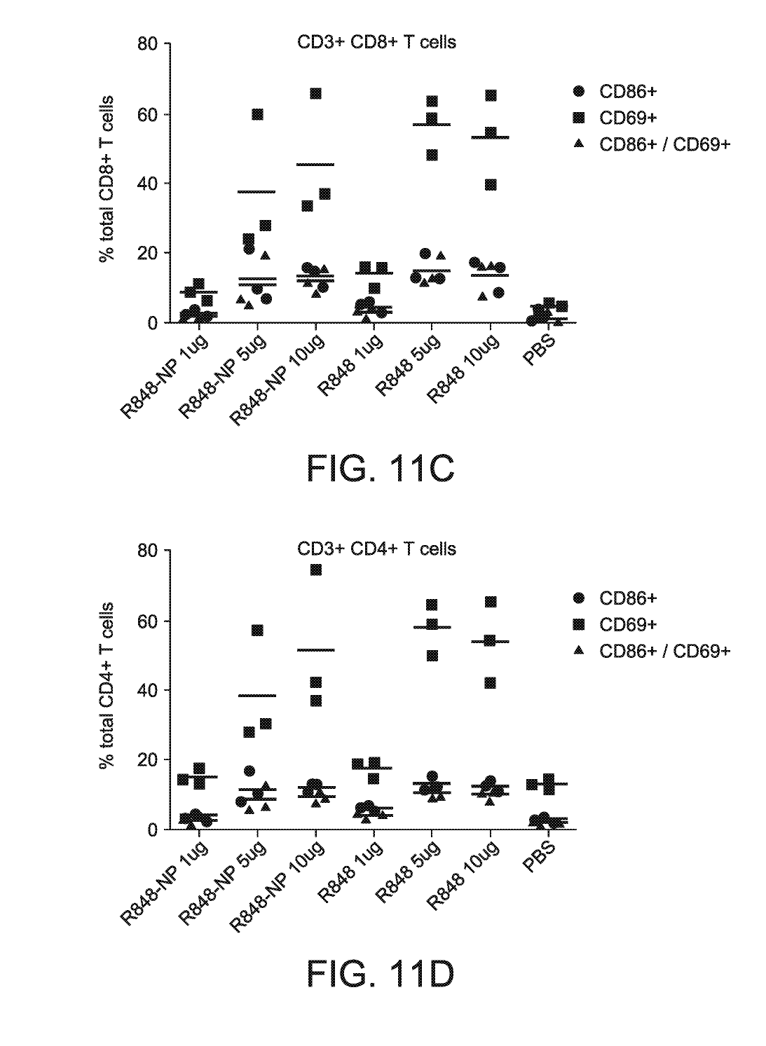

As demonstrated in the examples below, the immunotherapeutic small molecule adjuvant R848 loaded amph-NPs were injected subcutaneously to C57BL/6 mice. From there they disperse into the body and drain into the lymph system where they encounter cells sensitive to their adjuvants. Serum inflammatory cytokines were tested one hour post injection given the short half-life of small molecules. As shown in FIGS. 10A-10D, 5 μg of R848 delivered freely induced systemic cytokine production, which is considered undesirable systemic toxicity. However, 5 μg R848 delivered with amph-NPs induced minimal systemic toxicity. Importantly, doses above 5 μg activated DCs, B and T cells regardless of delivery method (FIGS. 11A-11B). Altogether, this experiment demonstrated an application to use amph-NPs to deliver small molecule R848 that have comparable therapeutic outcomes but with minimal systemic toxicity. Thus is some embodiments, amph-NPs are utilized to deliver an effective amount of a cargo or agent to cells in induce or increase a desired physiological change or outcome with little or no systemic toxicity.

The amph-NP can be used in broad range of applications including, but not limited to, vaccine and other adjuvant therapies, immunomodulation, and treatment of microbial infections, cancer, autoimmune disease, inflammation, and inflammatory disorders. Examples of using amph-NPs to enhance radiotherapy are discussed in Yang, et al., ACS Nano, 8(9):8992-9002 (2014).

A. Cancer

The amph-NP including an active agent cargo can be administered to a subject in an effective amount to treat cancer. The method can reduce tumor size or burden, or prevent tumor growth compared to, for example, an untreated control. In some embodiments, the amph-NP are utilized to treat the cancer directly by delivering a therapeutic active agent to, or preferably into the cytosol of a cancer cell. Suitable therapeutic agents are discussed above and generally include small molecule chemotherapeutic drugs. Such embodiments are particular useful for treating cancers of the lymphatic cancers, including, but not limited to lymphoma such as Hodgkin's Disease and Non-Hodgkin's Lymphoma, and secondary cancers of the lymph nodes (e.g., metastasis of a primary cancer such as those discussed in more detail below).

In some embodiments the amph-NP are utilized to treat the cancer indirectly, by modulating an immune response against the cancer. For example, the amph-NP can be used to deliver to T cells an active agent that enhance or prolongs the activation of T cells (i.e., increasing antigen-specific proliferation of T cells, enhancing cytokine production by T cells, stimulating differentiation ad effector functions of T cells and/or promoting T cell survival) or overcome T cell exhaustion or anergy, or any combination thereof. The active agent can be, for example, one that increases T cell activation or proliferation, or reduces T cell suppression. The examples below describe an exemplary embodiment in which amp-NP delivered a small molecule diacylglycerol kinases (DGKs) inhibitor into the cytosol of activated T cells in an effective amount to block PD-L1 mediated immunosuppression.

The types of cancer that may be treated with the provided compositions and methods include, but are not limited to, the following: bladder, brain, breast, cervical, colo-rectal, esophageal, kidney, liver, lung, nasopharangeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

In some embodiments the particles are targeted to or otherwise used to deliver agents to PD-1+ T cells in tumor microenvironment. Delivery of small molecule immunostimulants or immune-suppression reverting drugs to PD-1 expressing cells (e.g., dysfunctional T cells) may increase therapy potency and decrease side effects the drugs.

B. Vaccines

A strategy to enhance vaccine potency is to improve the delivery of antigen and adjuvant molecules to critical antigen presenting cells (APCs) in secondary lymphoid organs (Jewell, et al., PNAS, 108(38):15745-50 (2011). Following traditional vaccine injection in peripheral tissues, soluble proteins or small particles (<50 nm) drain directly to lymph nodes, while cell-associated antigen or larger antigen particles access lymph nodes by APC uptake and trafficking). Intralymph node (i.LN) vaccination—injection of antigens/adjuvants directly into lymph nodes—has shown great promise for vaccine delivery, improving the potency of DNA, RNA, peptide, protein, and dendritic cell-based vaccines Senti, et al., Curr Opin Allergy Clin Immunol, 9:537-543 (2009)). Studies have demonstrated as much as 10⁶-fold reductions in antigen dose, 100-fold reductions in adjuvant dose, and enhanced protection with reduced side effects relative to traditional parenteral immunizations (Johansen, Eur J Immunol, 35:568-574 (2005), and Maloy, et al., PNAS, 98:3299-3303 (2001)).

Vaccine and immunogenic compositions including amph-NPs, and methods of use thereof are provided. For vaccine and immunogenic related applications, amph-NPs can be loaded with an adjuvant or small molecule immunomodulator (e.g., an active agent that is immunostimulatory, or prevents immunosuppression). Additionally or alternatively the amph-NPs can include a peptide attached that can serve as an antigen.

For example in some embodiments, amph-NPs loaded with an adjuvant or small molecule immunomodulator are administered to the subject alone or in combination with an antigen, for example, a peptide antigen to which the immune response is desired. The antigen can be free or soluble, or can be attached to the same or different amph-NPs. Thus an immunogenic composition can include an effective amount of amph-NPs loaded with an adjuvant or small molecule immunomodulator to induce or enhance an immune response. In some embodiments, the immunogenic composition includes two or more different species of amph-NPs, for example two or more different adjuvants and/or small molecule immunomodulators.

In some embodiments, an antigen is attached to amph-NPs and administered to the subject alone or in combination with loaded with an adjuvant or small molecule immunomodulator. Thus an immunogenic composition can include an effective amount of antigen-attached amph-NPs to induce or enhance an immune response. The adjuvant or small molecule immunomodulator can be free or soluble, or entrapped by the same or different amph-NPs. In some embodiments, the immunogenic composition includes two or more different species of amph-NPs, for example, two or more different antigen-attached.

Amp-NPs can be utilized to deliver both the adjuvant or small molecule immunomodulator and the antigen. For example, one species of amph-NP can be used to entrap an adjuvant or small molecule and a different species of amph-NP can be attached with a peptide antigen attached thereto. The different species of amph-NP can have the same or different cores, SAMs, ligands, targeting moieties, etc. In some embodiments, the same amph-NP molecule includes both an entrapped adjuvant or small molecule and an attached peptide antigen.

The components of a vaccine, for example, adjuvant and antigen can be part of the same admixture or administered as separate compositions. The separate compositions can be administered through the same route of administration or different routes of administration.

Adjuvants

In some embodiments, the amph-NPs loaded with, for example, an adjuvant or small molecule immunomodulator are administered alone or in combination an antigen. The antigen, such as those discussed in more detail below, can be free or soluble or attached to an amph-NP.

Treatment with immune cell-activating adjuvant without coadministered antigen is emerging as an alternative approach to promote adaptive immune responses against endogenously produced tumor antigens that might simultaneously boost global immune cell activation status and dampen immune regulation (see, for example, Thomas, et al., Biomaterials, 35:814-824 (2014), and references cited therein particularly in the Introduction). Thomas also describes the secondary lymphoid tissues such as the lymph nodes as sites for targeted immunotherapy. DCs are present in high numbers in lymph nodes relative to peripheral tissues such as the skin, indicating that delivery of antigen and adjuvant to the lymph nodes might enhance vaccine efficiency. However, in addition to being a primary site for initiation of effector immune responses, the lymph nodes can be a prime site for induction of immune tolerance, because regulatory T (Treg) cells require the lymph nodes for activation. Additionally, lymphatic transport of antigen from the periphery to the draining lymph nodes has been implicated in tolerance induction against peripherally encountered antigens, such as tissue specific self-antigens being regionally drained to and through the tumor draining lymph nodes.

The compositions and methods can be utilized in vaccination protocols not only to induce prophylactic effector immunity, but also to modulate endogenous immune responses and redirect tolerogenic pro-tumor immune responses. Therefore, in some embodiments, the compositions and methods are used to induce an immunostimulatory response against an existing cancer or infection, or protect against a future cancer or infection. In some embodiments, the compositions and methods are utilized to reduce an overactive or inappropriate immune response such in during chronic inflammatory, autoimmune disease, rejection of grafts, etc. In particular embodiments, the amph-NPs are loaded with a small molecule adjuvant such as paclitaxel (PXL, a TLR-4 agonist as reported by Byrd, et al., Eur. J. Immunol., 31:2448-57 (2001)), and administered to a subject in need thereof alone or in combination with an antigen. In some embodiments, the compositions are used to prime immune cells ex vivo for cell mediated vaccines (see, e.g., Brussel, et al., Autoimmunity Reviews, 13(2):138-150 (2014)).

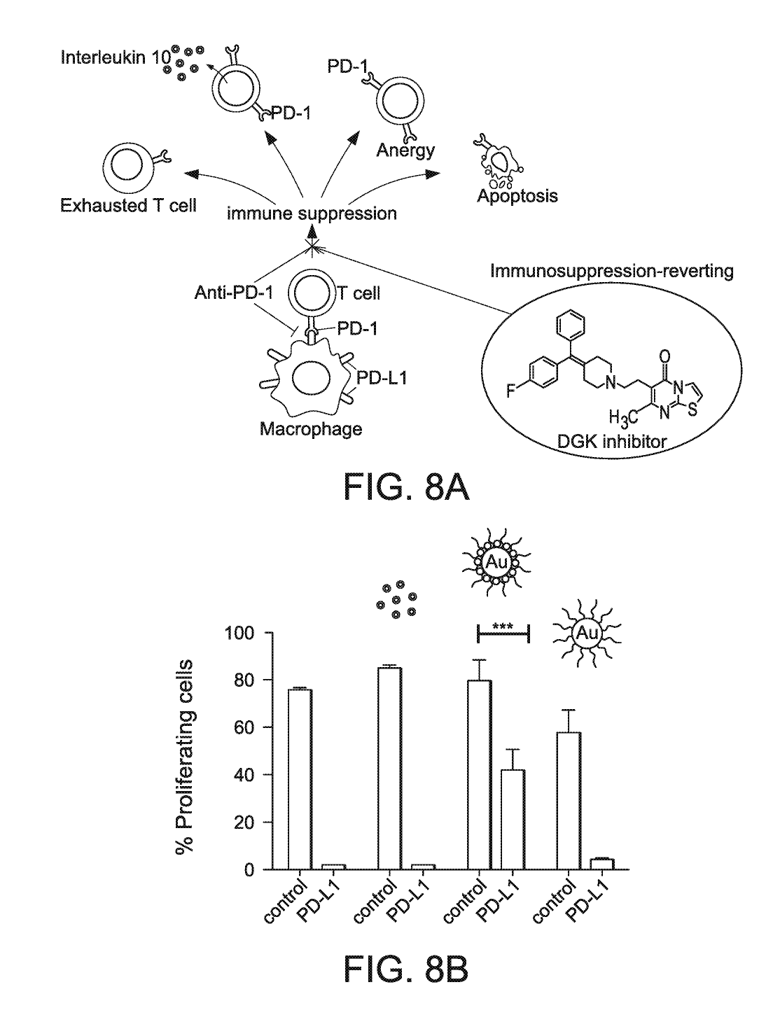

FIG. 8A is a diagram of macrophage-induced immunosuppression of T cells, and illustrates how DGK inhibitors can block the suppressive pathway prior to T cell dysfunction (adapted from thelancet.com/infection Vol. 13 Mar. 2013). The results of Amp-NP delivery of a DGKi to immune cells is shown in FIG. 8B.

Administration is not limited to the treatment of an existing tumor or infectious disease but can also be used to prevent or lower the risk of developing such diseases in an individual, i.e., for prophylactic use. Potential candidates for prophylactic vaccination include individuals with a high risk of developing cancer, i.e., with a personal or familial history of certain types of cancer. FIGS. 12C-12D show that amph-NP with a peptide antigen attached thereto can induces expression of pro-inflammatory cytokines and prevent tumor growth.

The compositions may be administered in conjunction with prophylactic vaccines, which confer resistance in a subject to subsequent exposure to infectious agents, or in conjunction with therapeutic vaccines, which can be used to initiate or enhance a subject's immune response to a pre-existing antigen, such as a tumor antigen in a subject with cancer, or a viral antigen in a subject infected with a virus.

The desired outcome of a prophylactic, therapeutic or de-sensitized immune response may vary according to the disease, according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease. For example, the stimulation of an immune response against a cancer may be coupled with surgical, chemotherapeutic, radiologic, hormonal and other immunologic approaches in order to affect treatment.

Antigens

In some embodiments, amph-NPs with an antigen attached, for example a peptide antigen, are administered alone or in combination an adjuvant or immunomodulator.

Antigens that can be used in the vaccine compositions can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof.

Suitable antigens are known in the art and are available from commercial government and scientific sources. In one embodiment, the antigens are whole inactivated or attenuated organisms. These organisms may be infectious organisms, such as viruses, parasites and bacteria. These organisms may also be tumor cells. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

Exemplary amph-NP loaded with adjuvants and immunomodulators are discussed above and in the Examples below. Other adjuvants that can be used in the disclosed compositions include oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

C. Infections

In some embodiments, the amph-NPs are utilized to deliver an anti-microbial active agent for treatment of a disease or infection. Lymph nodes filter the lymph, can carry bacteria, viruses, or other microbes. At sites of infection, large numbers of these microbial organisms collect in the regional lymph nodes and produce the local swelling and tenderness typical of a localized infection. Enlarged and occasionally confluent lymph nodes (lymphadenopathy) are often referred to as the “swollen glands” associated with diagnosis of infection. Depending on their location in the body, swollen lymph nodes are visible or identifiable by touch.

The amph-NP can be used to treat infection by, for example, delivering to infected lymph nodes, a small molecule drug that can reduce an infection, by for example, killing the microorganism or an infected cell carrying the microorganism. The infection can be, for example, a viral infection, bacterial infection, fungal, or protozoa infection. Thus, some embodiments provides a method for treating infection by administering to a subject an amount of an active agent-load Amph-NP to reduce one or more symptoms of the activity in the subject.

D. Inflammation, Inflammatory Disorders, and Autoimmune Disease

In some embodiments, the amph-NPs are utilized to deliver agent for treatment of inflammation, an inflammatory disorder, an autoimmune disease, transplant rejection, graft vs. host disease, or to reduce or otherwise treat lymphadenopathy. Lymphadenopathy, is enlarged, swollen, or tender lymph nodes, can be a sign of infection and or an autoimmune diseases such as systemic lupus erythematosus, rheumatoid arthritis, or sarcoidosi.

In some embodiments, the agent delivered is one that reduces an overactive or inappropriate immune response. For example, evidence indicates that regulatory T cells, particularly CD4+CD25+ regulatory T cells (CD4+ Treg cells) play an important role in the immunopathogenesis of autoimmune diseases, tumors, and organ transplantation (Wei, et al., Blood, 2006 Jul. 15; 108(2): 426-431.). The cross-talk between Treg cells and targeted cells, such as antigen-presenting cells (APCs) and T cells, is crucial for ensuring suppression by Treg cells in the appropriate microenvironment. Thus, in some embodiments, the agent is an immunomodulatory agent that reduces the number or activity of immune cells or increases the number or activity of immunosuppressive cells such as regulatory T cells. In some embodiments the agent increases the secretion, level, activity, etc. of an anti-inflammatory cytokine such as IL-10 or reduces the secretion, level, or activity a pro-inflammatory cytokine such as TNF-α.

In some embodiments, the subject to be treated is a transplant recipient, or subject with an inflammatory or autoimmune disease or disorder such as rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

E. Diagnostics and Imaging

In some embodiments, the Amph-NP are loaded with an imaging agent, and used to imaging of the lymph nodes. The Amph-NP can be used, for example, to identify or mark lymph nodes in a subject, and to characterize or diagnosis lymph node-related maladies including cancer, infections, autoimmune disease, etc.

The present invention will be further understood by the following non-limiting examples.

### EXAMPLES

### Example 1: Amph-NPs Localize to Lymph Nodes and Penetrate Cells

Materials and Methods

Synthesis of Amph-NPs

As described by Verma, et al. Nat. Mater. 2008, 7, 588-595, 0.9 mmol of HAuCl₄ (Sigma-Aldrich) was dissolved in 200 mL of ethanol, 0.9 mmol of the desired thiol ligand mixture was added while the reaction solution was stirred, and then a saturated ethanol solution of sodium borohydride (NaBH₄) was added dropwise for 2 h. The solution was stirred for 3 h, and the reaction vessel was then placed in a refrigerator overnight; precipitated particles were collected via vacuum filtration with quantitative filter paper. The residue was washed with ethanol, methanol, and acetone and dried under vacuum. To completely remove unbound ligands, particles were dialyzed using 5 in. segments of cellulose ester dialysis membrane (Pierce, MWCO 3500) that were placed in 1 L beakers of Milli-Q water and stirred slowly. The beakers were recharged with fresh DI water ca. every 8 h over the course of 72 h. The NP solutions were collected from the dialysis tubes, and the solvent was removed under vacuum at <45° C.

Nanoparticles of equivalent core sizes (2-4 nm) were tested for lymphatic transport and accumulation as a function of ligand chemistry (see FIG. 1). Four types of NPs were tested: Lipid membrane embedding amph-NPs composed of MUS or mixed MUS and OT ligands (Nano Letters 2013, 13 (9), 4060-4067); PEG(4CH) NPs composed of tetra-ethylene glycols with carboxylic end groups; and PEG 3k NPs composed of three kilodalton (kd) polyethylene glycols. See FIG. 1.

MUS and MUSOT amph-NPs are loaded with small molecule hydrophobic drugs. The hydrophobic regions in the NP ligand shells allow for an energetically favorable temporary storage location for small hydrophobic molecules (see FIG. 2). Upon delivery to the target cells, trans-membrane passage results in disturbance of the ligand shell and release of some cargo.

Amph-NP were prepared as described by Yang, et al., ACS Nano, 8(9):8992-9002 (2014). Amph-NP surfaces are protected by monolayers of mixed hydrophobic ligands (octanethiol) and hydrophilic ligands (11-carbon alkanethiol terminated with a sulfonate group).

Fluorescence Dye Labeling of Amph-NPs

To track gold nanoparticles by fluorescence microscopy, amph-NPs were labeled with a thiolated BODIPY dye as previously described (Verma, et al., Nat. Mater., 7:588-595 (2008)). Briefly, 5 μL of BODIPY-SH (2.45 mg/mL in 2:1 water/dimethylformamide mixture) was added to 10 mg of gold nanoparticles in 1 mL of water. The solution was covered with foil to protect it from light and agitated at speed of 750 rpm on a shaker for 3-4 days at 25° C. Unconjugated BODIPY-SH was completely removed by topping up the eppendorf with acetone and centrifuging at 14 kg for 2 min (repeated four times). Excess acetone was evaporated in a vacuum oven overnight. The dried nanoparticles were dissolved in water, and nanoparticle concentrations were determined by reading the absorbance at 520 nm.

The MUS and OT ligands protecting the gold core span approximately 1.6 nm, resulting in a total hydrodynamic diameter of 5.4 nm that is similar to the thickness of a lipid bilayer (4-5 nm).

Injection into Animals to Assess Tissue Localization

To characterize the extent of lymph node targeting achieved by a s.c. nanoparticle injection as a function of ligand shell chemistry, groups of C57Bl/6 mice were injected with MUSOT, MUS, PEG (4CH) or PEG3k coated gold particles of the same core size (˜2-4 nm) on one site subcutaneously near the tail base, and collected local and distal LNs from the left and right flanks of the animals 24 hr later.

Test the biodistribution of amph-NPs, amph-NPs were injected intravenously or subcutaneously into mice. The organs of interest were collected 3 hours or 24 hours post injection and quantified by inductively coupled plasma atomic emission spectroscopy (ICP-AES).

To determine if the amphiphilic ligand shell is what gives these gold nanoparticles such a unique biodistribution, hydrophilic gold nanoparticles that are protected by shorter ligands (3-carbon alkanethiols terminated with a sulfonate group) were synthesized. The hypothesis is that shorter ligand protected NPs of same core size may not favor membrane-embedding due to insufficient hydrophobic-hydrophobic interactions.

Cell Penetration

To test whether MUSOT NPs could cross epithelium barrier of mouse lungs, C57BL/6J mice were given intratracheal injections of BODIPY-MUSOT or saline. Their lungs were collected, stained with CD326, CD11b, CD11c, F4/80, CD64, B220, CD3, CD4, CD8, NK1.1, Ly6C, Ly6G, and analyzed by CyTOF and flow cytometry.

Results

Experimental and computer simulation results showed that these highly water soluble amph-NPs penetrate and embed in lipid membranes due to their unique surface chemistry and conformation.

Tissue Localization

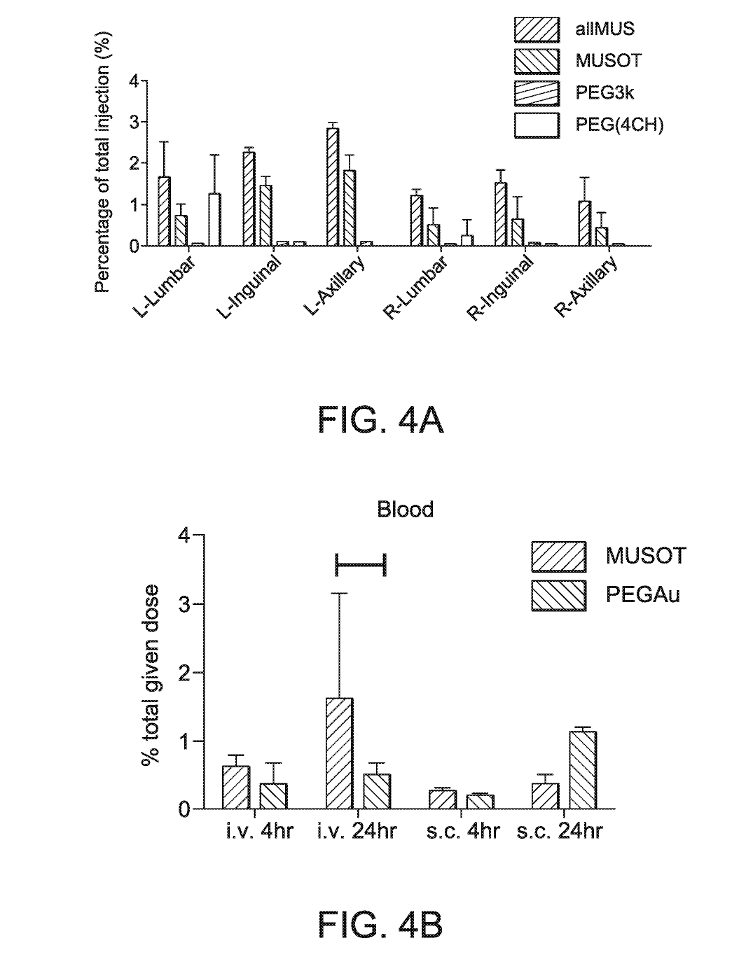

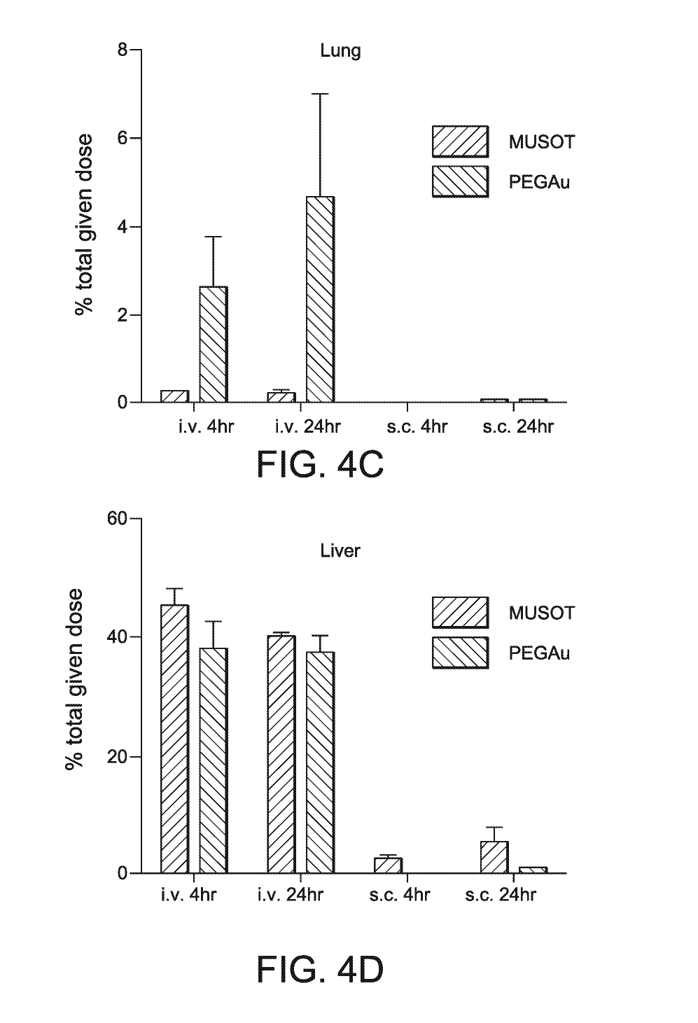

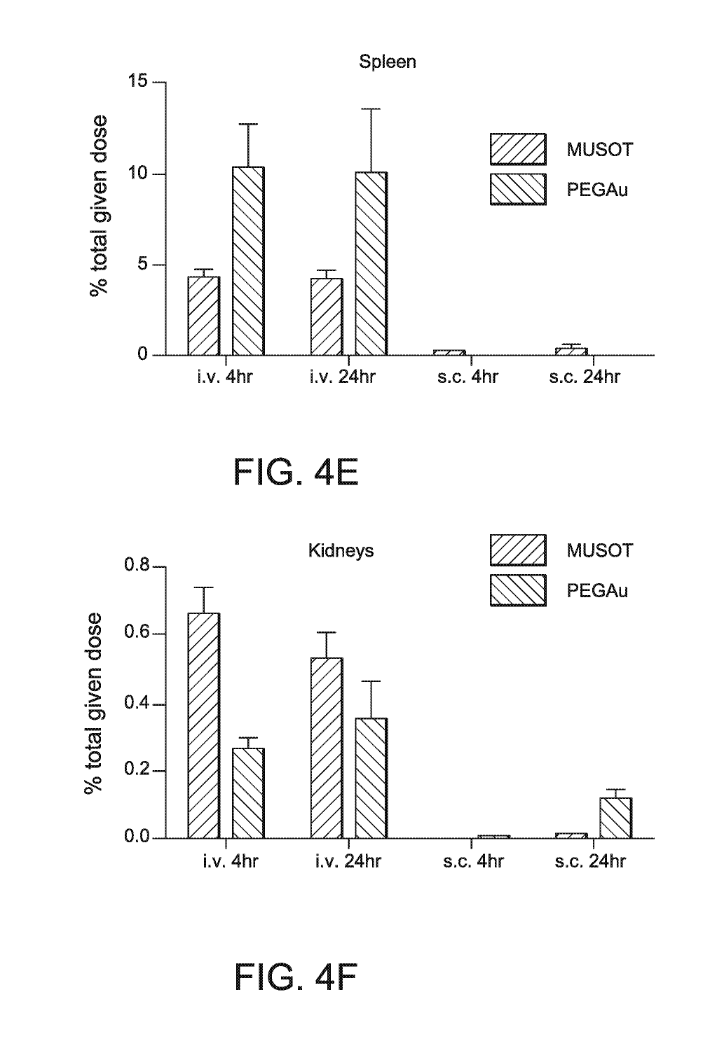

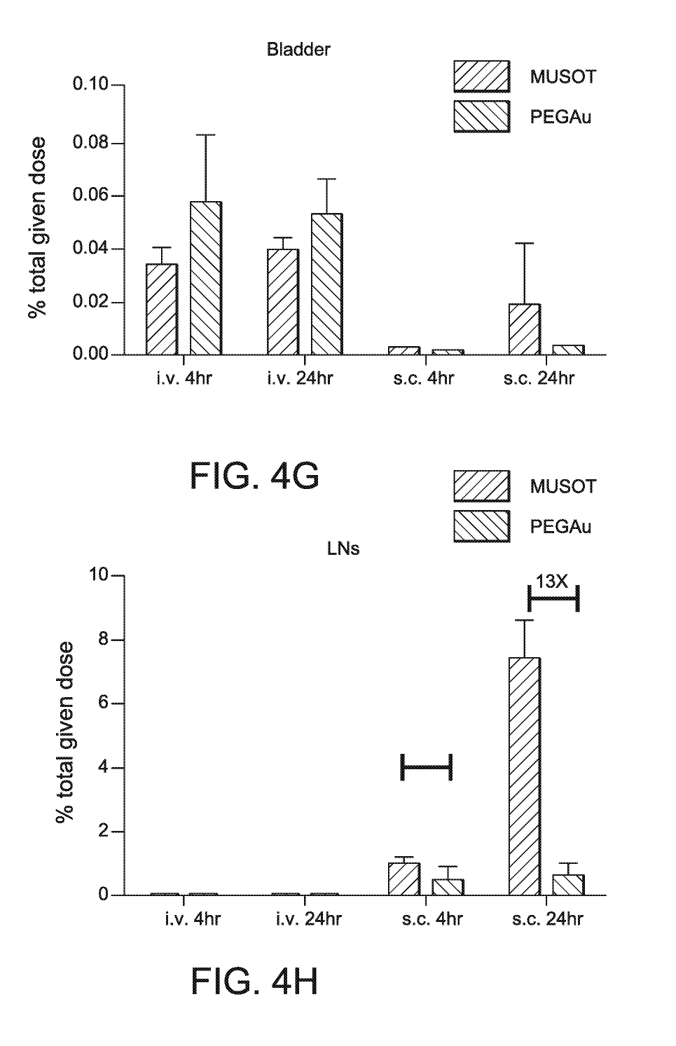

FIG. 4A is a graph of ICP-AES quantification (percentage of total injection) of Au NPs in lymph nodes 24 h post single site s.c. tail base injection as a function of ligand bound to the NPs: allMUS, MUSOT, PEG3k, and PEG(4CH). Au NPs solubilized in PBS and 50 μL of 6 mg/mL were injected subcutaneously on the left side of tail base. Lymph nodes were collected 24 h post injection and analyzed by inductively coupled plasma atomic emission spectroscopy (ICP-AES). FIG. 4B is a graph of the percent total administered dose comparing levels of MUSOT Amph-NPs in the blood, which were four times higher than the control PEG-Au. FIG. 4C-4G shows the organ distribution of the MUSOT Amph-NPs in spleen, kidney, liver, lung and bladder, demonstrating the Amph-NPs were not filtered by the lung. FIG. 4H shows that the MUSOT is 13× higher in the lymph nodes compared to PEGylated NPs. FIGS. 5A-5B look at distribution of nanoparticles in natural killer cells (NK), T cells, B cells, macrophages, dendritic cells and neutrophils.

24 hr following a single subcutaneous injection near the base of the tail in mice, a striking 5-10% of the total injection dose was found in the draining lymph nodes. ICP-AES quantification of gold in the tissues showed that amph-NPs (MUS or MUSOT) not only dispersed to the local (left flank) inguinal, lumbar, and axillary lymph nodes, but also reached the contralateral (right flank) nodes (FIG. 4A). Summing the uptake across all of these nodes, MUS amph-NPs showed the greatest accumulation with 10% of the injected dose accumulated at 24 hr.

By contrast, control PEG(4CH)NPs injected into a single site accumulated only at the nearest draining LN (L-lumbar) with minimal dispersion to either distal or contralateral sites, and PEG3KNPs had less than 0.5% of total injection in the local site, and an undetectably low number of NPs in all other LNs.

Altogether amph-NPs accumulated in LNs 12-fold more than control PEG-NPs, providing evidence of the amphiphilic ligand surface chemistry's importance for this LN targeting effect.

The biodistribution of amph-NPs via both i.v. and s.c. injection revealed that the gold nanoparticles strikingly accumulated in lymph nodes to ˜8-fold higher levels than all other tissues collected (spleen, liver, and kidney) in terms of mass amph-NPs per mg tissue. This lymph node tropic accumulation is promising for immunomodulatory drug delivery because the majority of lymphocytes reside in lymph nodes (LNs).

Cell Penetration

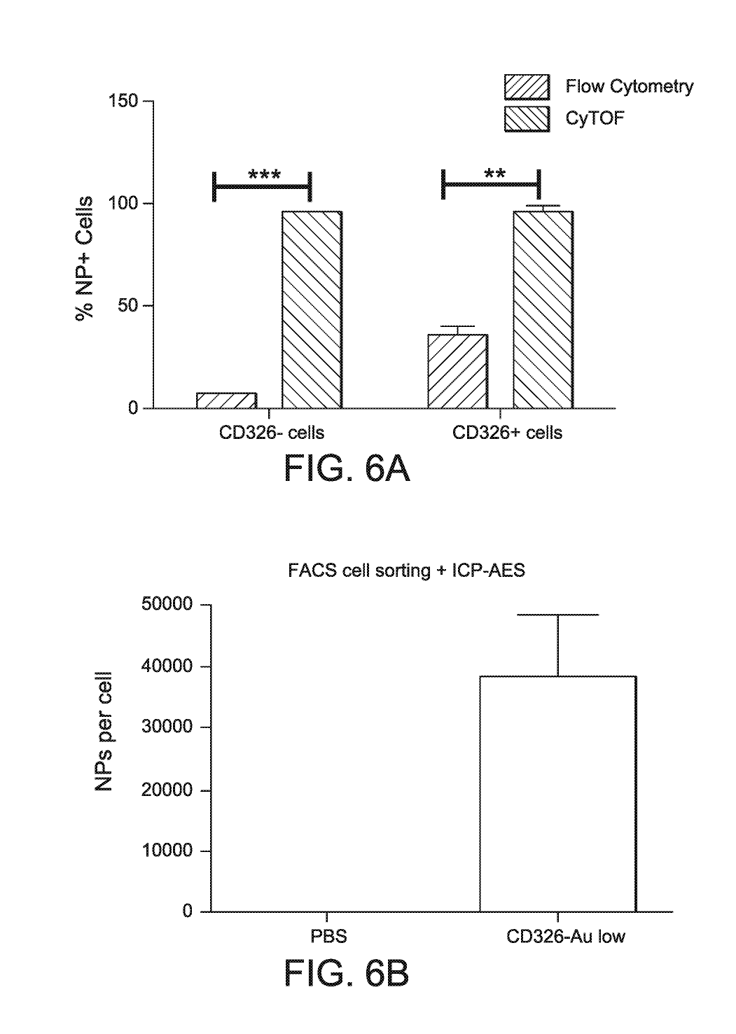

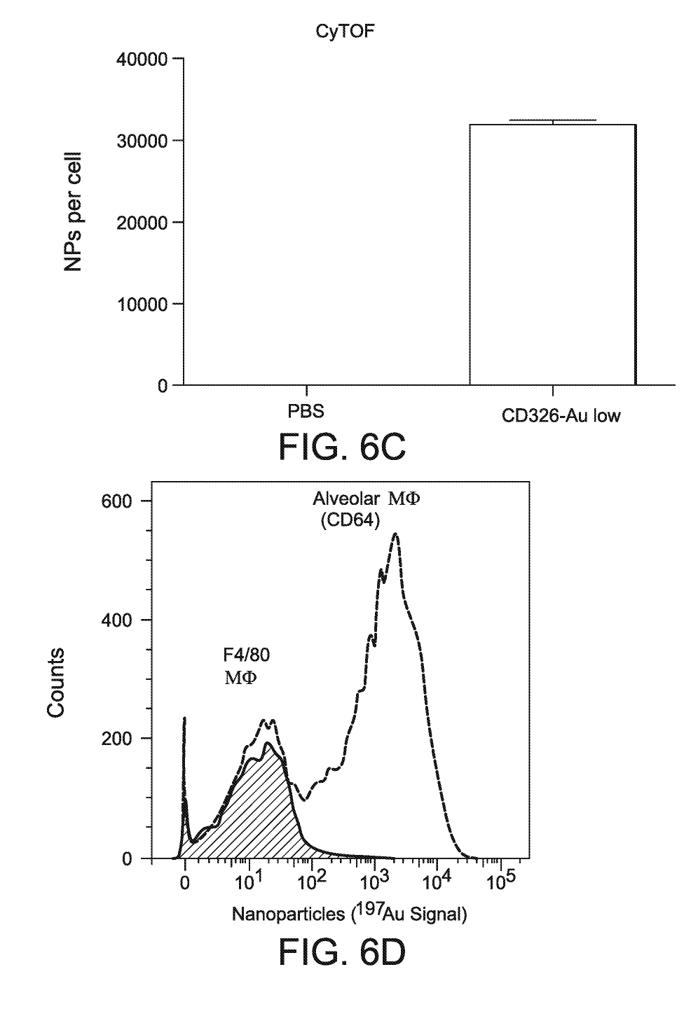

CyTOF analysis indicated that 24 h post i.t. injection, nearly 100% of lung cells (regardless of CD326- or CD326+) were infiltrated by amph-NPs (FIG. 6A). The data inconsistency between CyTOF and Flow cytometry analyses is caused by auto fluorescence background in cells which decreased signal to noise ratio.

To show that flow cytometer-determined BODIPY negative “Au low” cell populations actually did contain gold particles, these “Au low” cells were FACS-sorted and analyzed by ICP-AES. As shown in FIG. 6B, the number of gold particles per cell is in close agreement with CyTOF data (FIG. 6C). Among all cell types tested, alveolar macrophages (MΦ) in the lung contained the highest concentration of MUSOT amph-NPs. Gold concentration in alveolar MΦs was over 100-fold higher than F4/80 MΦs (FIG. 6D). Collectively, these results show that amph-NP can effectively penetrate epithelium and be used to target alveolar macrophages in the lungs.

### Example 2: Drug Loading and Delivery to T Cells In Vitro

Materials and Methods

Drug Loading

In this drug loading strategy, DGKi or other hydrophobic drugs in organic solvent are mixed with an aqueous suspension of amph-NPs and dialyzed to remove the organic solvent, leading to partitioning of the drug into the hydrophobic pockets of the amph-NP ligand shell. This approach allowed one to load drugs that are nearly insoluble in aqueous solutions to high degrees of “solubility” via incorporation into MUS or MUSOT amph-NPs.

More detailed procedures to prepare small molecules-loaded NPs are as follows: Hydrophobic small molecules are dissolved in pure ethanol, and mixed with NPs in a dialysis tube against water. Dialysis tubes containing 100-500 Da molecular weight cut off allows ethanol to permeate while retains most small molecules. As ethanol gradually removed from NP drug mixture, hydrophobic small molecules are driven into hydrophobic ligand shells of NPs to minimize unfavorable interaction with water. Loaded drugs were then quantified via absorbance using UV-vis spectrometer.

To test immunomodulatory drug loading capacity, small-molecule drug N-acetylcysteine (NAC) was loaded directly onto the gold core via sulfur-gold covalent bonding. The lymphocyte-modulating hydrophobic small molecule diacylglycerol kinase inhibitor (DGKi) was also phased into the hydrophobic monolayers of amph-NPs by a solvent replacement method. The drug loading efficiency was determined by HPLC and UV-vis.

In this drug loading strategy, DGKi or other hydrophobic drugs in organic solvent are mixed with an aqueous suspension of amph-NPs and dialyzed to remove the organic solvent, leading to partitioning of the drug into the hydrophobic pockets of the amph-NP ligand shell. Hydrophobic small molecules are dissolved in pure ethanol, and mixed with NPs in a dialysis tube against water. Dialysis tubes containing 100-500 Da molecular weight cut off allows ethanol to permeate while retains most small molecules. As ethanol gradually removed from NP drug mixture, hydrophobic small molecules are driven into hydrophobic ligand shells of NPs to minimize unfavorable interaction with water. Loaded drugs were then quantified via absorbance using UV-vis spectrometer. This approach allowed one to load drugs that are nearly insoluble in aqueous solutions to high degrees of “solubility” via incorporation into MUS or MUSOT amph-NPs.

To determine what factors regulate this mode of drug loading, drug loading as a function of gold core diameter and ligand composition was assessed. Three different small molecules were tested: DGK inhibitors, R848 and TGF-beta inhibitors.

The DGK inhibitor (DGKi) has the structure:

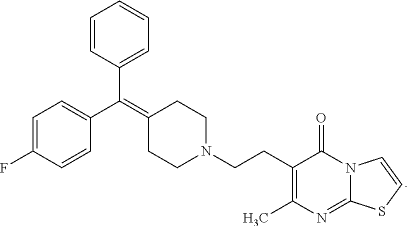 [SMILES: Cc1nc2sccn2c(=O)c1CCN1CCC(=C(c2ccccc2)c2ccc(F)cc2)CC1]

The imidazoquinoline compound R848 (Resiquimod) is a guanosine derivative and an agonist for TLR7 and TLR8. T848 has the structure:

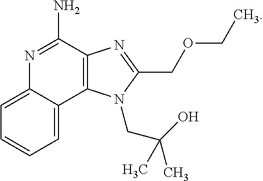 [SMILES: CCOCc1nc2c(N)nc3ccccc3c2n1CC(C)(C)O]

The TGF-beta inhibitor 5525334 (also referred to as TGFbi) has the structure:

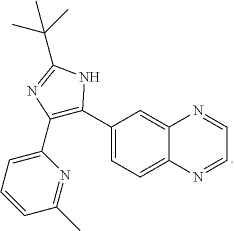 [SMILES: Cc1cccc(-c2nc(C(C)(C)C)[nH]c2-c2ccc3nccnc3c2)n1]

Results

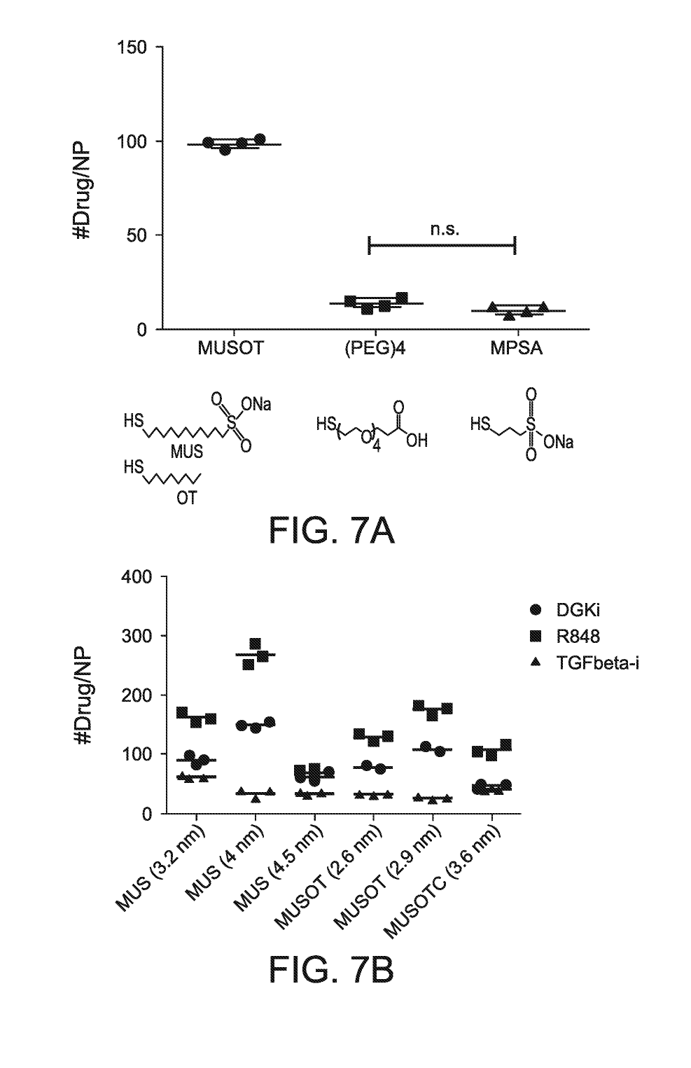

FIGS. 7A-7B are graphs quantifying drug loading capacity: DGKi loading in MUSOT, PEG(4CH) and MPSA NPs is shown in FIG. 7A. Drug loading of small molecules DGKi, R848, and TGF-beta inhibitor loading in amph-NPs of different core sizes is shown in FIG. 7B.

The effect of ligand length on drug loading, assuming that long hydrophobic ligands are crucial for drug loading, was compared. FIG. 7A showed that short hydrophobic ligand (MPSA)-coated gold NPs with only three hydrocarbons have 10-fold decreased loading capacity compared to MUSOT amph-NPs. PEGylated gold NPs with a hydrophilic poly(ethylene glycol) ligand shell also resulted in low drug loading, as expected for the proposed mechanism of hydrophobic drug sequestration.

It appears that 4 nm MUS NPs have optimal loading for both DGKi and R848, while 3.2 nm MUS NPs have optimal loading capacity for TGFbeta-i.

### Example 3: Lymph Node Targeted Amph-NPs can Modulate Immune Responses

Materials and Methods

Cytotoxic T cells were incubated with αCD3/CD28 activating antibody and either control IgG, or with PD-L1 IgG (PD-1 agonist antibody) to induce T cell suppression, and further treated with vehicle, DGK inhibitor alone, DGK inhibitor loaded amph-NP, or amph-NP alone.

To demonstrate that amph-NP drug delivery platform enhances cytosolic concentration of small molecule drugs, TGF-beta inhibitor S525334 was loaded to amph-NP ligand shells incubated with mouse CD8⁺ T cells for 1 hour at 37° C. Drugs delivered to T cells were extracted and analyzed by HPLC.

Results

PD-L1 binding to PD-1 can induce an intracellular signaling cascade that leads to T cell suppression, including T cell exhaustion and T cell anergy. Diacylglycerol kinases (DGKs), which negatively regulate Ras activity, are upregulated in anergic and exhausted T cells. Although this is desirable when trying to resolve an immune response, it is not desirable when it happens during certain chronic infections or acute conditions such as sepsis and in cancers (FIG. 8A). DGK inhibitors can be used to block DGK, and therefore block the T cell suppression pathway, leaving T cells active (e.g., proliferative). However, DGK inhibitors have to be delivered to the lipid membranes and/or the cytosol of the T cells to be effective.

FIG. 8B is a graph of the % proliferating cells with PD-L1 or control IgG treated with vehicle, diacyl kinase inhibitor, Amph-NPs delivering diacyl kinase inhibitor, and Amph-NP alone. The IgG controls show high proliferation in all treatment groups (71.3%, 89.9%, 94.0%, and 70.7%) because the cells were not treated with PD-L1 and therefore not suppressed. The PD-L1 treatment groups show low proliferation (e.g., T cell suppression) in no treatment (0.98%), drug alone (2.71%), and nanoparticle alone (1.78%), but near normal proliferation when treated with drug-loaded nanoparticles (72.0%—third graph from the right on the bottom). Amph-NP-carried DGKi reversed 60% of dysfunctional T cells. This shows that the nanoparticles can be used to deliver a small molecule drug into the cytosol of T cells in an effective amount to prevent or reverse PD-L1-induced immunosuppression.

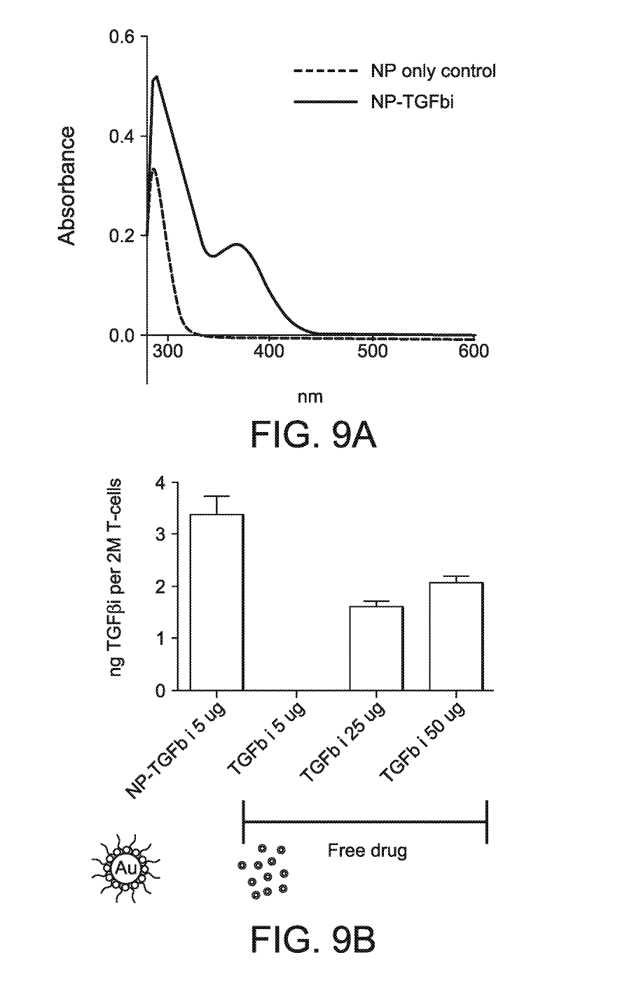

FIGS. 9A-9B show that TGF-beta inhibitor delivered with amph-NPs significantly enhanced cytosolic drug concentration in CD8+ T cells compared to free drug at the same dose.

### Example 4: LN-Targeted Amph-NPs Activated Immune Cells in LNs while Reducing Systemic Toxicity In Vivo

Materials and Methods

R848 (Resiquimod, a TLR7/8 agonist) was loaded into amph-NPs, and administered subcutaneously at the tail base. Serum was collected 1 hour post injection to evaluate systemic toxicity. Cells in LNs were isolated 24 h post injection to analyze immune cell activation.

Results

Small molecules like R848 diffuse quickly into blood vessels, which may lead to severe systemic adverse effect. An experiment was designed to determine if by using lymph node targeted NPs, systemic adverse effects could be reduced while activation of local lymphocytes remains effective. Systemic toxicity was compared by measuring TNF, IL-6, IL-10, and MCP-1, and measuring activation of lymphatic cells.

FIGS. 10A-10D are dot plots of pg/ml for 10, 5, and 1 μg TNF (10A), IL-6 (10B), IL-10 (10C), and MCP-1 (10D), showing reduced systemic toxicity via nanoparticle drug delivery system.

FIGS. 11A-11D are dot plots of showing the activation of dendritic cells (11A) and B cells and T cells: B220+CD3-cells (11B), CD3+CD8+ T cells (11C), CD3+CD4+ T cells (11D) via R848 (1, 5, 10 μg) delivered freely or with NPs.

### Example 5: LN-Targeted Amph-NPs Enhance Peptide Vaccine Delivery

Materials and Methods

Peptide Conjugation and Quantification

SIINFEKL (SEQ ID NO:1) peptide constructs were custom synthesized by LifeTein with the following structure: (N terminus) FITC-aminohexanoic acid (Ahx)-SIINFEKL-Ahx-cysteamide (C terminus) (SEQ ID NO:2) (FIG. 12A), with purity >95%. Lyophilized peptide was dissolved in DMF at 1 mg mL⁻¹. A mass ratio of gold:peptide of 4:1 in DMF was mixed in a glass vial and placed on a shaker to allow coupling reaction for 4 days.

1000 μg of gold NPs (10 mg/mL stock concentration in water) were mixed with 250 μg of peptides (SEQ ID NO:2) (1 mg/mL stock concentration in DMF). Therefore, the final reaction mixture contains ˜28.5% water and 71.5% DMF. Peptide conjugation was based on ligand place exchange (thiol gold chemistry).

To remove uncoupled peptide, the MUS/OT-peptide solution was first diluted in water (<5% DMF) and spun at 3500 rpm for 15 minutes in an amicon 10 kDa MWCO centrifugal tube. The above-mentioned washing step was performed repeatedly for a total of four times. To quantify peptide conjugation efficiency, 20 μL beta-mercaptoethanol (14.3M stock solution) and 20 μL DMF were added to an aliquot (0.1 mg in 60 μL H₂O) of purified MUSOT-peptide conjugates and allowed to react for 48 hours on a shaker at 25° C. Peptide conjugation efficiency was determined by fluorescence readout of FITC at excitation of 488 nm and emission of 520 nm using a standard curve made using uncoupled MUSOT particles doped with known amounts of peptide construct subjected to the same reaction conditions. The mass ratio of conjugated peptide to gold was determined to be ˜51 μg peptide per mg gold, which corresponds to ˜9 peptide constructs per NP.

Vaccine Studies

Eight week old female C57BL/6 mice were immunized (primed on day 1, boosted on day 14) with 8 μg of CpG (ODN 1826 VacciGrade, InvivoGen) mixed with SIINFEKL (SEQ ID NO:1) peptide (10 μg peptide conjugated-AuNP, 10 μg free peptide, 50 μg free peptide or 10 μg free peptide construct). Vaccines were formulated in 100 μL sterile saline with half of the volume injected subcutaneously on either side of the tail base. To monitor antigen-specific T-cells, mice were bled, and blood samples were processed as follows: 100 μL of blood was incubated with 500 μL ACK lysis buffer at 25° C. for 5 minutes followed by centrifugation, then this process was repeated for a second round of lysis. Cells were incubated in tetramer staining buffer (PBS, 1% BSA, 5 mM EDTA, 50 nM dasatinib), Fc block, and OVA tetramer (iTAg Tetramer/PE-H-2K^{b} OVA, MBL) in the dark for 45 minutes at 25° C. Anti-CD8a (53-6.7) APC antibody (1:200) was added to cell solutions and incubated for an additional 15 minutes at 4° C. Cells were washed twice in flow cytometry buffer containing 100 nM DAPI, and run on a BD FACS LSR Fortessa. Data was analyzed using FlowJo.

Tumor Challenge

B16-OVA cells were a kind gift from Dr. Glenn Dranoff at the Dana-Farber Cancer Institute. B16-OVA cells were cultured in complete DMEM (DMEM supplemented with 10% FBS, 100 units mL⁻¹ penicillin, 100 μg mL⁻¹ streptomycin, and 4 mM L-alanyl-L-glutamine), maintained at 37° C. and 5% CO₂, and passaged when 70-80% confluent. A challenge of 2.5×10⁵ B16-OVA cells was injected subcutaneously on the right flank of previously immunized mice in 50 μL of sterile saline. Tumor size was measured (longest dimension×perpendicular dimension) three times weekly, and an area was calculated by multiplying these dimensions. Mice were euthanized when tumor area exceeded 100 mm². All animal work was conducted under the approval of the Massachusetts Institute of Technology (MIT) Division of Comparative Medicine in accordance with federal, state, and local guidelines.

Intracellular Cytokine Staining

PBMCs were isolated from immunized mice and cultured in RPMI supplemented with 10% FBS, 100 units mL⁻¹ penicillin, 100 μg mL⁻¹ streptomycin, and 4 mM L-alanyl-L-glutamine with 10 μg mL⁻¹ SIINFEKL peptide. After 2 hours, Brefeldin A (1/1000, eBiosciences) was added to inhibit cytokine secretion. After 6 hours total incubation with peptide, cells were washed, stained extracellularly with anti-CD8a (53-6.7, eBioscience), fixed and permeabilized (BD Cytofix/Cytoperm), and stained intracellularly with anti-IFN-γ (XMG1.2, eBioscience) and anti-TNF-α (MP6-XT22, eBioscience). Cells were run on a BD FACS LSR Fortessa and data was analyzed using FlowJo.

Results

MUS/OT-mediated peptide delivery greatly increased the potency of the peptide vaccination, eliciting at peak ˜6-fold more CD8⁺ T-cells than the equivalent dose of free SIINFEKL peptide (SEQ ID NO:1), and greater than a 5-fold higher dose of free peptide or immunization with free FITC-SIINFEKL-linker construct (SEQ ID NO:2) (FIG. 12B). MUS/OT-peptide-vaccinated mice challenged with ovalbumin-expressing B16F10 melanoma tumor cells at day 150 exhibited robust cytokine-producing CD8⁺ T-cell responses, and these animals were fully protected from tumor outgrowth, in contrast to free peptide-immunized controls (FIGS. 12C-12D). This example illustrates the power of single-cell inorganic NP analysis coupled with multiparameter phenotyping to develop new nanomedicines.

### CONCLUSIONS

Collective, data presented above demonstrates the amph-NPs are highly water-soluble, membrane-interactive, and home to lymph nodes efficiently. These amph-NPs are effective drug carriers for cytosolic delivery of immune system-relevant drugs to enhance immunity.

1. Amphiphilic nanoparticles (Amph-NPs) comprising:
metal cores surrounded by a self-assembled monolayer formed from a plurality of thiol-containing ligands, wherein the monolayer is capable of embedding within and traversing lipid membranes and trafficking to the lymph nodes; and
one or more active agents entrapped within the monolayer, one or more peptides attached thereto, or a combination thereof;
wherein the nanoparticles are for use in vaccine and other adjuvant therapies, immunomodulation, or for treatment of microbial infections, cancer, autoimmune disease, inflammation and inflammatory disorders.

2. The nanoparticles of claim 1, wherein the metal core comprises a metal selected from the group consisting of gold, silver, platinum, palladium, copper, and aluminum.

3. The nanoparticles of claim 1, wherein the ligands are selected from the group consisting of mercaptopropionic acid, mercapto undecanoic acid, 4-amino thiophenol, hexanethiol, octanethiol, decanethiol, duodecanethiol, salts thereof, and mixtures thereof.

4. The nanoparticles of claim 1, wherein the ligands are selected from the group consisting of 11-mercaptoundecanesulfonic acid and salts thereof, 3-mercaptopropane-1-sulfonic acid and salts thereof, octanethiol, and mixtures thereof.

5. The nanoparticles of claim 1, wherein the ligands are comprised of an anchor group-tether group-end group structure; wherein the anchor group is or contains a thiol.

6. The nanoparticles of claim 5, wherein the end group is functionalized with an agent.

7. The nanoparticles of claim 6, wherein the agent is a polypeptide.

8. The nanoparticles of claim 6, wherein the monolayer comprises a combination of the agent functionalized ligands and ligands selected from the group consisting of 11-mercaptoundecanesulfonic acid and salts thereof, octanethiol, and mixtures thereof.

9. The nanoparticles of claim 5, wherein the end group is selected from the group consisting of —SO₃⁻, —C(O)O⁻, —SO₂⁻, —OSO₃⁻², —SOO⁻, —CH₃, —SO₃H, —COOH, —PO₄³⁻, PO₃²⁻, —PO₃H⁻, —PO₃H₂, and NHₓR₄₋ₓ⁺ (wherein x is 1-4).

10. The nanoparticles of claim 5, wherein the tether group is a saturated or unsaturated, optionally substituted linear or branched alkyl group, preferably in the range of C₃-C₂₀.

11. The nanoparticles of claim 5, wherein the anchor group is a thiol (—SH).

12. The nanoparticles of claim 1, wherein the core comprises gold.

13. The nanoparticles of claim 1, wherein the active agents are selected from the group consisting of a hydrophobic therapeutic, prophylactic or diagnostic agent.

14. The nanoparticles of claim 1, further comprising a targeting moiety for lymph node tissue, natural killer cells, dendritic cells, subcapsular sinus macrophages, T cells, or B cells.

15. A pharmaceutical composition comprising the amphiphilic nanoparticles of claim 1.

16. The pharmaceutical composition of claim 15, wherein the nanoparticle comprises one or more active agents entrapped within the monolayer.

17. The pharmaceutical composition of claim 16, further comprising an antigen.

18. The pharmaceutical composition of claim 17, wherein the antigen is a peptide which is attached to the nanoparticle.

19. The pharmaceutical composition of claim 15, wherein the nanoparticle comprises one or more peptides attached thereto.

20. The pharmaceutical composition of claim 19, further comprising an adjuvant.

21. A pharmaceutical composition comprising:
a first species of amphiphilic nanoparticles comprising metal cores surrounded by a self-assembled monolayer formed from a plurality of thiol-containing ligands, wherein the monolayer is capable of embedding within and traversing lipid membranes and trafficking to the lymph nodes; and one or more active agents entrapped within the monolayer; and
a second species of amphiphilic nanoparticles comprising metal cores surrounded by a self-assembled monolayer formed from a plurality of thiol-containing ligands, wherein the monolayer is capable of embedding within and traversing lipid membranes and trafficking to the lymph nodes, and one or more peptides attached thereto;
wherein the first and second amphiphilic nanoparticles are for use in vaccine and other adjuvant therapies, immunomodulation, or for treatment of microbial infections, cancer, autoimmune disease, inflammation and inflammatory disorders.

22. A method of selectively delivering a therapeutic, prophylactic or diagnostic agent to lymph nodes, lymph node tissues, or cells in the lymph nodes comprising administering the individual Amph-NPs of claim 1.

23. The method of claim 22, wherein the therapeutic, prophylactic or diagnostic agent is for use in vaccine and other adjuvant therapies or immunomodulation.

24. The method of claim 22, wherein the therapeutic, prophylactic or diagnostic agent is for the treatment of microbial infection.

25. The method of claim 22, wherein the therapeutic, prophylactic or diagnostic agent is for the treatment of cancer.

26. The method of claim 22, wherein the therapeutic, prophylactic or diagnostic agent is for the treatment of autoimmune disease, inflammation or inflammatory disorders.


SEQUENCE LISTING (2 sequences)

SEQ ID NO: 1 - protein, 8 aa, Artificial Sequence
  Synthetic peptide
SIINFEKL

SEQ ID NO: 2 - protein, 8 aa, Artificial Sequence
  Synthetic peptide
  MISC_FEATURE (1)..(1): N-terminal conjugate Fluorescein isothiocyanate (FITC)-aminohexanoic acid (Ahx)-
  MISC_FEATURE (8)..(8): C-terminal conjugate -aminohexanoic acid (Ahx)-cysteamide
SIINFEKL